SEQUENCE LISTING (14 sequences)

SEQ ID NO: 1 - protein, 5 aa, synthetic construct
CGGHA

SEQ ID NO: 2 - protein, 5 aa, synthetic construct
CPGHA

SEQ ID NO: 3 - protein, 5 aa, synthetic construct
CGAHA

SEQ ID NO: 4 - protein, 5 aa, synthetic construct
CPAHA

SEQ ID NO: 5 - protein, 6 aa, synthetic construct
GCGGHA

SEQ ID NO: 6 - protein, 6 aa, synthetic construct
ACPGHA

SEQ ID NO: 7 - protein, 6 aa, synthetic construct
GCPGHA

SEQ ID NO: 8 - protein, 8 aa, synthetic construct
EDEDEAGG

SEQ ID NO: 9 - protein, 8 aa, synthetic construct
EGEGEAGG

SEQ ID NO: 10 - protein, 5 aa, synthetic construct
EGEGE

SEQ ID NO: 11 - protein, 19 aa, synthetic construct
EDEDEGAGGKGAGKGAGKG

SEQ ID NO: 12 - protein, 487 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKGSTSGSGKPGSGEGSTKGEVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSGGGASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKDYKCKVSNKALPAPIEKTISKAKGQPREPQVYTLPPSRDELTRNQVSLTCLVKGFYPSDIAVEWESNGQPENNYKTTPPVLDSDGSFFLYSKLTVDKSRWQQGNVFSCSVMHEALHNHYTQKSLSLSPGGGGACPGHA

SEQ ID NO: 13 - protein, 18 aa, synthetic construct
GSTSGSGKPGSGEGSTKG

SEQ ID NO: 14 - protein, 16 aa, synthetic construct
SLSLSPGGGGACPGHA

(19) United States
(12) Patent Application Publication
(10) Pub. No.: US 2024/0342298 A1
(43) Pub. Date: Oct. 17, 2024

(54) METHODS OF TREATING TUMORS BY USING MOLECULAR CONSTRUCT

(71) Applicant: Immunwork Inc., Taipei (TW)

(72) Inventors: Hsing-Mao CHU, Taipei City (TW); Yueh-Hsiang YU, Taipei City (TW); Wei-Ting TIAN, Taipei City (TW); Tse-Wen CHANG, Taipei City (TW); Wei-Chen LIN, Taipei City (TW); Shih-Syuan CHENG, Taipei City (TW)

(21) Appl. No.: 18/633,337

(22) Filed: Apr. 11, 2024

Related U.S. Application Data
(60) Provisional application No. 63/459,249, filed on Apr. 13, 2023.

(51) Int. Cl. A61K 47/68; A61P 35/00

(52) U.S. Cl. CPC A61K 47/6803; A61K 47/6849; A61K 47/6889; A61P 35/00

(57) ABSTRACT

Disclosed herein is a method of treating a tumor in a subject. The method comprises administering to the subject a molecular construct, which comprises an anti-CD38 antibody, and a plurality of lenalidomide molecules or hydrolyzed lenalidomide molecules linked to the anti-CD38 antibody. According to some embodiments of the present disclosure, the administration of the molecular construct gives rise to an effective amount of the lenalidomide molecules or the hydrolyzed lenalidomide molecules that is at least 1,000 times less than an effective amount of the lenalidomide molecule used alone or in combination with the anti-CD38 antibody for the treatment of the tumor.

### CROSS-REFERENCE TO RELATED APPLICATIONS

This application relates to and claims the benefit of U.S. Provisional Application No. 63/459,249, filed Apr. 13, 2023; the content of the application is incorporated herein by reference in its entirety.

### SEQUENCE LISTING XML

The present application is being filed along with a Sequence Listing XML in electronic format. The Sequence Listing XML is provided as an XML file entitled P4326_US_SEQ_AF.txt, created Apr. 11, 2024, which is 14 Kb in size. The information in the electronic format of the Sequence Listing XML is incorporated herein by reference in its entirety.

### BACKGROUND OF THE INVENTION

### 1. Field of the Invention

The present disclosure in general relates to the field of tumor treatment. More particularly, the present disclosure relates to a method of treating tumors by using a molecular construct comprising an anti-CD38 antibody and a plurality of lenalidomide molecules or hydrolyzed lenalidomide molecules.

### 2. Description of Related Art

Multiple myeloma (MM), also known as myeloma or plasma cell myeloma, is a type of bone marrow cancer, in which cancerous plasma cells accumulate in the bone marrow and crowd out normal blood cells, resulting in an overabundance of monoclonal paraprotein (M protein; an abnormal antibody), destruction of bone, and displacement of other hematopoietic cell lines. Since cancerous plasma cells would affect different areas of the body, the symptoms and signs of multiple myeloma vary greatly among patients. Common symptoms and signs associated with multiple myeloma include, bone pain, bone fracture, spinal cord compression, anemia, repeated infections, hypercalcemia, unusual bleeding, thickened blood, fatigue, kidney problem, and neurological problem.

The RVd regimen, i.e., the combination of bortezomib (VELCADE®), lenalidomide (Revlimid®) and dexamethasone, is often used as the first-line treatment for multiple myeloma. According to the reports, more than 90% of patients with myeloma respond well to the treatment. However, the treatment merely decreases the number of cancerous cells in patient's bone marrow thereby alleviating the symptoms of multiple myeloma, without curing the underlying disease. Further, high doses of lenalidomide or other drugs in the combined regimen usually cause severe adverse effects (e.g., bleeding, difficult or labored breathing, thromboembolism, neutropenia, thrombocytopenia, fever, cramps, seizures, irregular heartbeat, speech and moving problems, and confusion). As most of the multiple myeloma patients are elders (age 50-70), the strong side effects raise concerns.

In view of the forging, there exists in the related art a need for a novel method for treating multiple myeloma.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to a method of treating a tumor in a subject. The method comprises administering to the subject a molecular construct, which comprises an anti-CD38 antibody, and a plurality of lenalidomide molecules or hydrolyzed lenalidomide molecules linked to the anti-CD38 antibody.

According to some embodiments of the present disclosure, the administration of the molecular construct gives rise to an effective amount of the lenalidomide molecules or hydrolyzed lenalidomide molecules that is at least 1,000 times less than an effective amount of the lenalidomide molecule used alone or in combination with the anti-CD38 antibody for the treatment of the tumor. In certain preferred embodiments, the effective amount of the lenalidomide molecules or hydrolyzed lenalidomide molecules is about 10,000 times less than the effective amount of the lenalidomide molecule used alone or in combination with the anti-CD38 antibody for the treatment of the tumor.

Preferably, the molecular construct is administered to the subject in an amount of about 0.01 to 100 mg/Kg body weight per dose; more preferably, about 0.1 to 10 mg/Kg body weight per dose. According to some preferred embodiments, the molecular construct is administered to the subject once every four weeks.

According to certain embodiments, the anti-CD38 antibody comprises a pair of CH2-CH3 segments of an immunoglobulin G (IgG), and a pair of anti-CD38 single-chain variable fragments (scFvs) respectively linked to the N-termini of the pair of CH2-CH3 segments. In these embodiments, the pair of CH2-CH3 segments comprises a plurality of linking residues independently selected from the group consisting of lysine (K) and cysteine (C) residues, and the plurality of lenalidomide molecules or hydrolyzed lenalidomide molecules are respectively linked to the plurality of linking residues.

According to alternative embodiments, the anti-CD38 antibody comprises a pair of CH2-CH3 segments of an IgG, a pair of anti-CD38 scFvs respectively linked to the N-termini of the pair of CH2-CH3 segments, and a pair of linking peptides respectively linked to the C-termini of the pair of CH2-CH3 segments. In these embodiments, the pair of linking peptides comprises a plurality of C residues, and the plurality of lenalidomide molecules or hydrolyzed lenalidomide molecules are respectively linked to the plurality of C residues of the pair of linking peptides.

Preferably, each of the linking peptides of the anti-CD38 antibody comprises the amino acid sequence of “CGGHA” (SEQ ID NO: 1), “CPGHA” (SEQ ID NO: 2), “CGAHA” (SEQ ID NO: 3), “CPAHA” (SEQ ID NO: 4), “GCGGHA” (SEQ ID NO: 5), “ACPGHA” (SEQ ID NO: 6), or “GCPGHA” (SEQ ID NO: 7). In one exemplary embodiment, each of the linking peptides of the anti-CD38 antibody comprises the amino acid sequence of “ACPGHA” (SEQ ID NO: 6).

According to some exemplary embodiments, each of the linking peptides of the anti-CD38 antibody consists of the amino acid sequence of “CGGHA” (SEQ ID NO: 1), “CPGHA” (SEQ ID NO: 2), “CGAHA” (SEQ ID NO: 3), “CPAHA” (SEQ ID NO: 4), “GCGGHA” (SEQ ID NO: 5), “ACPGHA” (SEQ ID NO: 6), or “GCPGHA” (SEQ ID NO: 7). In one specific example, each of the linking peptides of the anti-CD38 antibody consists of the amino acid sequence of “ACPGHA” (SEQ ID NO: 6).

Optionally, the molecular construct further comprises a linker unit configured to link the lenalidomide molecules or hydrolyzed lenalidomide molecules and the linking peptides. The linker unit in its structure comprises a center core and a plurality of linking arms, in which the center core comprises 2 to 10 K residues, at least one filler independently disposed between two K residues; and a terminal spacer having two termini, in which one of the termini is linked to the N-terminus of the first K residue or the C-terminus of the last K residue, and the other of the termini is linked to the C residue of the linking peptide of the anti-CD38 antibody. According to some optional embodiments of the present disclosure, one terminus of each linking arm is linked to one of the K residues of the center core, and the other terminus of each linking arm is linked to each lenalidomide molecule or hydrolyzed lenalidomide molecule.

According to various embodiments of the present disclosure, each of the filler and the terminal spacer independently comprises, (1) 1 to 12 non-K amino acid residues, or (2) a PEGylated amino acid having 1 to 12 repeats of ethylene glycol (EG) unit. Preferably, the terminal spacer comprises at least three negative charged amino acid residues. In some exemplary embodiments, the terminal spacer comprises the amino acid sequence of “EDEDEAGG” (SEQ ID NO: 8), “EGEGEAGG” (SEQ ID NO: 9) or “EGEGE” (SEQ ID NO: 10). According to one specific example, the center core comprises the amino acid sequence of “EDEDEGAGGKGAGKGAGKG” (SEQ ID NO: 11).

The linking arm comprises 2-12 non-K amino acid residues, a polyethylene glycol (PEG) chain having 2-24 repeats of EG units, or a combination thereof. In one exemplary embodiment, the linking arm comprises a valine-alanine (Val-Ala)dipeptide and a PEG chain having 3 repeats of EG units.

In certain embodiments, each of the linking arms is linked to the &-amino group of the K residue.

The tumor treatable with the present method may be a solid tumor or a diffused tumor. Examples of the solid tumor include, but are not limited to, melanomas, esophageal carcinomas, gastric carcinomas, brain tumor, small cell lung cancer, non-small cell lung cancer, bladder cancer, breast cancer, pancreatic cancer, colon cancer, rectal cancer, colorectal cancer, renal cancer, hepatocellular carcinoma, ovary cancer, prostate cancer, thyroid cancer, testis cancer, head and neck squamous cell carcinoma, and a combination thereof. Exemplary diffused tumors include, but are not limited to, acute lymphocytic leukemia (ALL), chronic lymphocytic leukemia (CLL), acute myelogenous leukemia (AML), chronic myelogenous leukemia (CML), Hodgkin lymphoma, non-Hodgkin lymphoma (e.g., lymphoplasmacytic lymphoma, follicular lymphoma, diffuse large B-cell lymphoma, mantle cell lymphoma, or marginal zone lymphoma), multiple myeloma, and a combination thereof. According to one embodiment of the present disclosure, the tumor is multiple myeloma.

Many of the attendant features and advantages of the present disclosure will become better understood with reference to the following detailed description considered in connection with the accompanying drawings.

### BRIEF DESCRIPTION OF THE DRAWINGS

The present description will be better understood from the following detailed description read in light of the accompanying drawings, where:

FIGS. 1A to 1C are schematic diagrams respectively depicting the structures of the molecular constructs 10, 20, 30 according to embodiments of the present disclosure;

FIG. 2 is a schematic diagram depicting the structure of lenalidomide bundle according to Example 1 of the present disclosure;

FIG. 3 depicts the reversed-phase analytical high-performance liquid chromatography (HPLC) elution profile of the Mal-lenalidomide bundle according to Example 1 of the present disclosure;

FIG. 4 depicts the electrospray ionization-tandem mass spectrometry (ESI-MS) result of the Mal-lenalidomide bundle according to Example 1 of the present disclosure;

FIG. 5A is a schematic diagram depicting the structure of 2-chain (anti-CD38 scFv)-hIgG1.Fc-(Gly)₃-BM fusion protein according to Example 2 of the present disclosure.

FIG. 5B depicts the non-reducing sodium dodecyl sulfate polyacrylamide gel electrophoresis (SDS-PAGE) analysis of 2-chain (anti-CD38 scFv)-hIgG1.Fc-(Gly)₃-BM fusion protein according to Example 3 of the present disclosure;

FIG. 6A is a schematic diagram depicting the structure of native 2-chain (anti-CD38 scFv)-hIgG1.Fc-(Gly)₃-BM-lenalidomide bundle according to Example 4 of the present disclosure;

FIG. 6B depicts the non-reducing SDS-PAGE analysis of recombinant 2-chain (anti-CD38 scFv)-hIgG1.Fc-(Gly)₃-BM-lenalidomide bundles according to Example 4 of the present invention;

FIGS. 7A and 7B respectively depict the result of non-reducing SDS-PAGE analysis (FIG. 7A) and the elution profile of size-exclusive chromatography (FIG. 7B) of the stabilized 2-chain (anti-CD38 scFv)-hIgG1.Fc-(Gly)₃-BM-lenalidomide bundles according to Example 6 of the present invention;

FIGS. 8A and 8B are the results of HPLC and ESI-MS respectively depicting the release of lenalidomide from MM cells treated with the stabilized 2-chain (anti-CD38 scFv)-hIgG1.Fc-(Gly)₃-BM-lenalidomide bundles according to Example 9 of the present disclosure; Panel (A) of FIG. 8B: the ESI-MS profile of the released lenalidomide from the MM cells; Panels (B) to (D) of FIG. 8B: the ESI-MS results of two lenalidomide metabolites and lenalidomide respectively corresponding to peaks (1), (2) and (3) of the ESI-MS profile depicted in Panel (A) of FIG. 8B;

FIG. 9 depicts the matrix-assisted laser desorption/ionization time-of-flight (MALDI-TOF) result of the stabilized 2-chain (anti-CD38 scFv)-hIgG1.Fc-(Gly)₃-BM-lenalidomide bundles according to Example 10 of the present invention;

FIG. 10 is the result of enzyme-linked immunosorbent assay (ELISA) that depicts the binding affinities of the 2-chain (anti-CD38 scFv)-hIgG1.Fc-(Gly)₃-BM fusion protein (unconjugated) and the stabilized 2-chain (anti-CD38 scFv)-hIgG1.Fc-(Gly)₃-BM-lenalidomide bundles (stabilized conjugates) to human CD38-expressing cells according to Example 13 of the present invention;

FIG. 11 is the result of flow cytometry that depicts the binding abilities of the stabilized 2-chain (anti-CD38 scFv)-hIgG1.Fc-(Gly)₃-BM-lenalidomide bundles (stabilized conjugates) to H929 and U266-CD38 multiple myeloma (MM) cell lines according to Example 14 of the present disclosure;

FIGS. 12A to 12G respectively depict the in vitro cytotoxicity of specified drugs according to Example 16 of the present disclosure; FIGS. 12A to 12D: the percentage (%) of cell viability of H929 cells treated with stabilized conjugates (i.e., the stabilized 2-chain (anti-CD38 scFv)-hIgG1.Fc-(Gly)₃-BM-lenalidomide bundles), anti-CD38 monoclonal antibody (mAb), daratumumab, daratumumab combined with lenalidomide, or lenalidomide for 5 hours (FIG. 12A), 1 day (FIG. 12B), 3 days (FIG. 12C), or 5 days (FIG. 12D); FIGS. 12E to 12G: the percentage (%) of cell viability of MM.1S cells (FIG. 12E), U266-CD38-cells (FIG. 12F), and Daudi cells (FIG. 12G) treated with stabilized conjugates, anti-CD38 mAb, daratumumab, daratumumab combined with lenalidomide, or lenalidomide for 5 days;

FIG. 13 depicts the in vitro stability of specified drugs in human plasma according to Example 18 of the present disclosure, in which daratumumab, unconjugated (i.e., 2-chain (anti-CD38 scFv)-hIgG1.Fc-(Gly)₃-BM fusion protein), and conjugated Ab (stabilized) (i.e., the stabilized 2-chain (anti-CD38 scFv)-hIgG1.Fc-(Gly)₃-BM-lenalidomide bundles) were respectively dissolved in human plasma and incubated at 37° C. for 7, 14, 21 and 28 days, followed by the analysis of ELISA; stabilized ADC (total Ab): the products detected by HRP-conjugated anti-human IgG-Fc antibody; stabilized ADC (conjugated Ab): the products detected by anti-lenalidomide bundle antibody and anti-mouse IgG-Fc antibody;

FIG. 14 depicts of the anti-tumor effects of specified treatments in xenograft tumor model according to Example 20 of the present disclosure, in which the tumor-bearing mice (the average size of tumor being 115±15 mm³) were respectively administered with phosphate-buffered saline (PBS; serving as a control group), stabilized conjugates (i.e., the stabilized 2-chain (anti-CD38 scFv)-hIgG1.Fc-(Gly)₃-BM-lenalidomide bundles), and daratumumab (Dara); and

FIGS. 15A and 15B respectively depict the effects of specified treatments on inhibiting tumor size (FIG. 15A) and tumor weight (FIG. 15B) in xenograft tumor model according to Example 21 of the present disclosure, in which the tumor-bearing mice (the average size of tumor being 150±20 mm³) were respectively administered with PBS, stabilized conjugates (i.e., the stabilized 2-chain (anti-CD38 scFv)-hIgG1.Fc-(Gly)₃-BM-lenalidomide bundles), daratumumab (Dara), daratumumab combined with lenalidomide (Dara/Lena), and lenalidomide (Lena); ** P<0.01; *** P<0.001.

In accordance with common practice, the various described features/elements are not drawn to scale but instead are drawn to best illustrate specific features/elements relevant to the present invention. Also, reference numerals and designations in the various drawings are used to indicate elements/parts.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

In the present specification and claims, the term “antibody” is used in the broadest sense and covers fully assembled antibodies, antibody fragments that bind with antigens, such as antigen-binding fragment (Fab/Fab′), F(ab′)2 fragment (having two antigen-binding Fab portions linked together by disulfide bonds), variable fragment (Fv), single chain variable fragment (scFv), bi-specific single-chain variable fragment (bi-scFv), nanobodies (also referred to as single-domain antibodies, sdAb), unibodies and diabodies. An “antibody fragment” comprises a portion of an intact antibody, preferably the antigen-binding region or variable region of the intact antibody. An antibody fragment may comprise a pair of scFv fused to the N- or C-terminal of a pair of CH2-CH3 segments derived from human immunoglobulin (Ig). Typically, an “antibody” refers to a protein consisting of one or more polypeptides substantially encoded by immunoglobulin genes or fragments of immunoglobulin genes. The well-known immunoglobulin genes include the kappa, lambda, alpha, gamma, delta, epsilon, and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. A typical immunoglobulin (antibody) structural unit is known to comprise a tetramer, which is composed of two identical pairs of polypeptide chains, with each pair having one “light” chain (about 25 kDa) and one “heavy” chain (about 50-70 kDa). The N-terminus of each chain defines a variable region of about 100 to 110 or more amino acids primarily responsible for antigen recognition. The terms variable light chain (VL) and variable heavy chain (VH) refer to these light and heavy chains, respectively. According to some embodiments of the present disclosure, the antibody fragment can be produced by modifying the nature antibody or by de novo synthesis using recombinant DNA methodologies. In certain embodiments of the present disclosure, the antibody can be bispecific, and can be in various configurations. For example, a bispecific antibody may comprise two different antigen binding sites (variable regions). In various embodiments, bispecific antibodies can be produced by hybridoma technique or recombinant DNA technique.

As used herein, the terms “link,” “couple,” and “conjugate” are used interchangeably to refer to any means of connecting two components either via direct linkage or via indirect linkage between two components.

The terms “polypeptide” and “peptide” are used interchangeably to refer to a polymer having at least two amino acid residues. Typically, the polypeptide comprises amino acid residues ranging in length from 2 to about 200 residues; nonetheless, it also encompasses macromolecules that has more than 200 amino acid residues. Where an amino acid sequence is provided herein, L-, D-, or beta amino acid versions of the sequence are also contemplated. Polypeptides also include amino acid polymers in which one or more amino acid residues are an artificial chemical analogue of a corresponding naturally occurring amino acid, as well as to naturally occurring amino acid polymers. In addition, the term applies to amino acids joined by a peptide linkage or by other, “modified linkages,” e.g., where the peptide bond is replaced by an α-ester, a β-ester, a thioamide, phosphoramide, carbomate, hydroxylate, and the like.

The term “fragment crystallizable region” or “Fc region”, as used herein, refers to the tail region of an immunoglobulin that interacts with cell surface receptors called Fc receptors and/or some proteins of the complement system. In structure, the Fc region comprises, from N-terminus to C-terminus, at least a hinge region (a short sequence of the heavy chain that links the CH1 and CH2 domains), a CH2 domain (the second constant domain of the heavy chain) and a CH3 domain (the third constant domain of the heavy chain). An Fc region of an IgG1 antibody can, for example, be generated by digestion of an IgG1 antibody with papain.

“Percentage (%) sequence identity” with respect to any amino acid sequence identified herein is defined as the percentage of amino acid residues in a candidate sequence that are identical to the amino acid residues in the specific reference sequence, after aligning the sequences and introducing gaps, if necessary, to achieve the maximum percent sequence identity, and not considering any conservative substitutions as part of the sequence identity. Alignment for purposes of determining the percentage of sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two amino acid sequences was carried out by computer program Blastp (protein-protein BLAST) provided online by Nation Center for Biotechnology Information (NCBI). The percentage sequence identity of a given sequence A to a subject sequence B (which can alternatively be phrased as a given sequence A that has a certain % sequence identity to a given sequence B) is calculated by the formula as follows:

$$\frac{X}{Y} \times 100 \%$$

where X is the number of amino acid residues scored as identical matches by the sequence alignment program BLAST in that program's alignment of A and B, and where Y is the total number of amino acid residues in the subject sequence B.

In certain embodiments, conservative substitutions of the amino acids comprising any of the sequences described herein are contemplated. In various embodiments, one, two, three, four, or five different residues are substituted. The term “conservative substitution” is used to reflect amino acid substitutions that do not substantially alter the activity (e.g., biological or functional activity and/or specificity) of the molecule. Typically, conservative amino acid substitutions involve substitution one amino acid for another amino acid with similar chemical properties (e.g., charge or hydrophobicity). Certain conservative substitutions include “analog substitutions” where a standard amino acid is replaced by a non-standard (e.g., rare, synthetic, etc.) amino acid differing minimally from the parental residue. Amino acid analogs are derived synthetically from the standard amino acids without sufficient change to the structure of the parent, are isomers, or are metabolite precursors. In the present application, the amino acid residues (1) lysine, which contains an amine group in its sidechain, (2) cysteine, which contains a thiol group in its sidechain, (3) serine and threonine, which contain a hydroxyl group in their sidechain, and (4) aspartic acid and glutamic acid, which contain a carboxyl group in their sidechain, are considered four distinctive groups of amino acids. These four groups of amino acids each contain in their sidechains a unique functional group, which may be applied for conjugating to various chemical components. Non-natural amino acids, which contain the same functional groups in the sidechains may be substituted for similar purposes.

In certain embodiments, polypeptides comprising at least 80%, preferably at least 85% or 90%, and more preferably at least 95% or 98% sequence identity with any of the sequences described herein are also contemplated.

The term “PEGylated amino acid” as used herein refers to a polyethylene glycol (PEG) chain with one amino group and one carboxyl group. According to the embodiments of the present disclosure, the PEGylated amino acid has the formula of NH₂—(CH₂CH₂O)ₙ—CO₂H. In the present disclosure, the value of n ranges from 1 to 20; preferably, ranging from 2 to 12.

As used herein, the term “terminus” with respect to a polypeptide refers to an amino acid residue at the N- or C-end of the polypeptide. Regarding a polymer, the term “terminus” refers to a constitutional unit of the polymer (e.g., the polyethylene glycol of the present disclosure) that is positioned at the end of the polymeric backbone. In the present specification and claims, the term “free terminus” is used to mean the terminal amino acid residue or constitutional unit is not chemically bound to any other molecules.

The terms “application” and “administration” are used interchangeably herein to mean the application of the present molecular construct to a subject in need of a treatment thereof.

The term “treat”, “treating” or “treatment” as used herein includes preventative (e.g., prophylactic), curative or palliative treatment. In particular, the term “treat”, “treating” or “treatment” as used herein refers to the application or administration of the present molecular construct to a subject, who has a medical condition (e.g., a caner), a symptom associated with the medical condition, a disease or disorder secondary to the medical condition, or a predisposition toward the medical condition, with the purpose to partially or completely alleviate, ameliorate, relieve, delay onset of, inhibit progression of, reduce severity of, and/or reduce incidence of one or more symptoms or features of said particular disease, disorder, and/or condition. Treatment may be administered to a subject who does not exhibit signs of a disease, disorder, and/or condition, and/or to a subject who exhibits only early signs of a disease, disorder and/or condition, for the purpose of decreasing the risk of developing pathology associated with the disease, disorder and/or condition. Treatment is generally “effective” if one or more symptoms or clinical markers are reduced as that term is defined herein. Alternatively, a treatment is “effective” if the progression of a symptom, disorder or condition is reduced or halted.

The term “effective amount” as used herein refers to the quantity of the present molecular construct that is enough to yield a desired therapeutic response. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two, or more doses in a suitable form to be administered at one, two or more times throughout a designated time period. The specific effective or sufficient amount will vary with such factors as particular condition being treated, the physical condition of the patient (e.g., the patient's body mass, age, or gender), the type of subject being treated, the duration of the treatment, the nature of concurrent therapy (if any), and the specific formulations employed and the structure of the compounds or its derivatives. Effective amount may be expressed, for example, as the total mass of active component (e.g., in grams, milligrams or micrograms) or a ratio of mass of active component to body mass, e.g., as milligrams per kilogram (mg/kg) or nanomole per kilogram (nmol/kg). Persons having ordinary skills could calculate the human equivalent dose (HED) for the medicament (such as the present molecular construct) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The term “tumor” as used herein, refers to all neoplastic cell growth and proliferation, whether malignant or benign, and all pre-cancerous and cancerous cells and tissues. In the present specification and claims, the term “tumor” comprises solid tumors and diffused tumors.

The term “solid tumor” as used herein, denotes an abnormal mass of tissue that usually does not contain cysts or liquid areas. Different types of solid tumors are named for the type of cells that form them. Examples of solid tumors include, but are not limited to, sarcomas and carcinomas. Generally, “sarcomas” are cancers arising from connective or supporting tissues such as bone or muscle. “Carcinomas” are cancers arising from glandular cells and epithelial cells, which line body tissues.

The term “diffused tumor” as used herein refers to leukemia and/or hematological malignancy that is formed from hematopoietic (blood-forming) cells and affect blood, bone marrow, or lymph nodes. The example of the diffused tumor includes, but is not limited to, acute lymphocytic leukemia (ALL), chronic lymphocytic leukemia (CLL), acute myelogenous leukemia (AML), chronic myelogenous leukemia (CML), Hodgkin lymphoma, non-Hodgkin lymphoma, and myeloma.

The terms “subject” as used herein refers to an animal including the human species that is treatable by the molecular construct and/or method of the present invention. The term “subject” intended to refer to both the male and female gender unless one gender is specifically indicated. Accordingly, the term “subject” comprises any mammals, which may benefit from the treatment method of the present disclosure. Examples of a “subject” include, but are not limited to, a human, rat, mouse, guinea pig, monkey, pig, goat, cow, horse, dog, cat, bird and fowl. In an exemplary embodiment, the subject is a human.

### II. DESCRIPTION OF THE INVENTION

In a conventional antibody-drug conjugate (ADC) construct, the antibody is a very large macromolecule with molecular weight of about 150,000 daltons, and the drug molecules (payloads) are small compounds with molecular weight in the range of several hundred daltons. The typical dosage of an ADC is in the range of 100 mg to several hundreds of mg. Thus, for a therapeutic drug to be applicable for combining with an antibody for the construction of an ADC, which exhibits cytotoxic or immunoregulatory activity or those mediating other types of effects on tumors, the potency of the drug must be very high, with IC₅₀ in the sub-nanomolar range (See, for example, David Dahlgren et al., Antibody-Drug Conjugates and Targeted Treatment Strategies for Hepatocellular Carcinoma: A Drug-Delivery Perspective; Molecules (2020), 25, 2861; or Alain Beck et al., Strategies and challenges for the next generation of antibody-drug conjugates; Nature Reviews Drug Discovery (2017), 16:315-337)). For example, the microtubule disrupting agents (e.g., monomethyl auristatin E (MMAE), monomethyl auristatin F (MMAF), mertansine (DM-1) and cribulin), the topoisomerase I inhibitors (e.g., camptothecin, exatecan and SN38), and DNA synthesis inhibitors (e.g., pyrrolobenzodiazepine (PBD) and its derivatives) cause lethal effects on the target cells at sub-nanomolar ranges, i.e., with EC₅₀ at the sub-nanomolar range. Lenalidomide, a thalidomide analogue, is a drug used clinically in several disease indications, most notably, multiple myeloma, usually in combination of other drugs. It is taken orally at dosages of 20-40 mg per day for most days during the treatment. In various studies, lenalidomide is found to have an EC₅₀ at the 3 μM to 30 μM range (See, for example, A Lopez-Girona et al., Cereblon is a direct protein target for immunomodulatory and antiproliferative activities of lenalidomide and pomalidomide; Leukemia (2012), 26:2326-2335). Thus, it is well-accepted that lenalidomide is not suitable to be employed as a therapeutic drug in an ADC construct.

The present disclosure is based, at least in part, on the discovery that an ADC construct employing lenalidomide as the conjugated drug exhibits a therapeutic effect on reducing or eliminating tumor growth. According to the embodiments of the present disclosure, the conjugation of anti-CD38 antibody to lenalidomide molecule significantly improves the therapeutic effect of the lenalidomide molecule on tumors (e.g., multiple myeloma); compared to typical lenalidomide treatment schedule involving a 28-day-cycle, during which the lenalidomide molecule is administered daily until either disease progression or unacceptable toxicity, a single dose of the combined treatment (i.e., the bioconjugate of anti-CD38 antibody and lenalidomide molecule) provides a satisfactory effect on suppressing tumor growth in a subject, that avoids repeated administrations of the lenalidomide molecule and thus greatly reduces its adverse effect.

Accordingly, the present disclosure provides a method of treating a tumor in a subject. The method comprises administering to the subject a molecular construct, which comprises an anti-CD38 antibody, and a plurality of lenalidomide molecules or hydrolyzed lenalidomide molecules linked to the anti-CD38 antibody.

According to some embodiments of the present disclosure, the molecular construct comprises a plurality of lenalidomide molecules linked to the anti-CD38 antibody. In these embodiments, the administration of the molecular construct gives rise to an effective amount of the lenalidomide molecules that is at least 1,000 times less than an effective amount of the lenalidomide molecule used alone or in combination with the anti-CD38 antibody (i.e., a combined treatment, in which the lenalidomide molecule and anti-CD38 antibody are separately administered to the subject), for the treatment of the tumor; for example, the effective amount of the lenalidomide molecules may be 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, 11,000, 12,000 or more times less than the an effective amount of the lenalidomide molecule used alone or in combination with the anti-CD38 antibody for achieving the therapeutic purpose. According to some preferred embodiments, the effective amount of the lenalidomide molecules is about 10,000 times less than the effective amount of the lenalidomide molecule used alone or in combination with the anti-CD38 antibody. In one exemplary embodiment, the effective amount of the lenalidomide molecules is about 10,640 times less than the effective amount of the lenalidomide molecule alone used alone or in combination with the anti-CD38 antibody.

According to some embodiments of the present disclosure, the molecular construct comprises a plurality of hydrolyzed lenalidomide molecules linked to the anti-CD38 antibody. In these embodiments, the administration of the molecular construct gives rise to an effective amount of the hydrolyzed lenalidomide molecules that is at least 1,000 times less than an effective amount of the lenalidomide molecule used alone or in combination with the anti-CD38 antibody, for the treatment of the tumor; for example, the effective amount of the hydrolyzed lenalidomide molecules may be 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, 11,000, 12,000 or more times less than the an effective amount of the lenalidomide molecule used alone or in combination with the anti-CD38 antibody for achieving the therapeutic purpose. According to some preferred embodiments, the effective amount of the hydrolyzed lenalidomide molecules is about 10,000 times less than the effective amount of the lenalidomide molecule used alone or in combination with the anti-CD38 antibody. In one exemplary embodiment, the effective amount of the hydrolyzed lenalidomide molecules is about 10,640 times less than the effective amount of the lenalidomide molecule alone used alone or in combination with the anti-CD38 antibody.

According to some embodiments, the lenalidomide molecule has the structure of

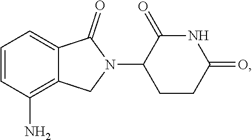 [SMILES: Nc1cccc2c1CN(C1CCC(=O)NC1=O)C2=O]

and the hydrolyzed lenalidomide molecule has the structure of

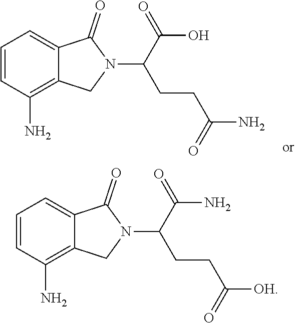

According to certain embodiments, the subject is a mouse. In these embodiments, the molecular construct is administered in an amount of about 1 nmol/Kg to 1,000 nmol/Kg (about 0.12 mg/Kg to 120 mg/Kg) body weight per dose; for example, the present molecular construct may be administered in an amount of about 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 nmol/Kg (alternatively, about 0.1, 0.11, 0.12, 0.13, 0.14, 0.15, 0.16, 0.17, 0.18, 0.19, 0.2, 0.25, 0.3, 0.35, 0.4, 0.45, 0.5, 0.55, 0.6, 0.65, 0.7, 0.75, 0.8, 0.85, 0.9, 0.95, 1, 2, 3, 4, 5, 6, 7, 8, 9, 10, 15, 20, 25, 30, 35, 40, 45, 50, 55, 60, 65, 70, 75, 80, 85, 90, 95, 100, 110, or 120 mg/Kg) body weight per dose for achieving the therapeutic purpose. Preferably, the molecular construct is administered in an amount of about 1 nmol/Kg to 100 nmol/Kg (about 0.12 mg/Kg to 12 mg/Kg) body weight per dose. More preferably, the molecular construct is administered in an amount of about 10 nmol/Kg to 50 nmol/Kg (about 1.2 mg/Kg to 6 mg/Kg) body weight per dose. In some exemplary embodiments, about 20 nmol/Kg (about 2.3 mg/Kg to 2.4 mg/Kg) body weight per dose of the present molecular construct is sufficient inhibit tumor growth in the subject.

A skilled artisan may readily determine the human equivalent dose (HED) of the present molecular construct, based on the doses determined from animal studies provided in working examples of this application. Accordingly, the effective amount of the present molecular construct suitable for use in a human subject may be in the range of about 0.08 nmol/Kg to 100 nmol/Kg (about 0.01 mg/Kg to 12 mg/Kg) body weight per dose; for example, about 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nmol/Kg (alternatively, about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, or 12 mg/Kg) body weight per dose. Preferably, the molecular construct is administered in an amount of about 0.8 nmol/Kg to 83 nmol/Kg (about 0.1 mg/Kg to 10 mg/Kg) body weight per dose. More preferably, the molecular construct is administered in an amount of about 0.8 nmol/Kg to 33 nmol/Kg (about 0.1 mg/Kg to 4 mg/Kg) body weight per dose. The dose can be administered in a single aliquot, or alternatively in more than one aliquot. The skilled artisan or clinical practitioner may adjust the dosage or regime in accordance with the physical condition of the patient or the severity of the diseases.

Preferably, the molecular construct is administered to the subject once every four weeks. In certain examples, the molecular construct is administered to the subject at an interval of four weeks for several times (e.g., for 2, 3, 4, 5, 6, 7, 8, 9, 10 or more times), until a desired therapeutic effect is achieved. As could be appreciated, a skilled artisan may adjust the dosage or regime in accordance with the physical condition of the patient or the severity of the diseases.

According to certain embodiments of the present disclosure, the molecular construct is in a form of an antibody-drug conjugate (ADC), which, as known in the art, is typically composed of a monoclonal antibody (mAb) covalently attached to a therapeutic drug (e.g., a cytotoxic drug). Specifically, in the embodiments, the anti-CD38 antibody of the molecular construct has a conventional structure, i.e., comprising a pair of heavy chain and a pair of light chain, in which each heavy chain comprises, form N-terminus to C-terminus, a heavy chain variable (VH) domain, a CH1 domain (the first constant domain of the heavy chain), a hinge domain, a CH2 domain (the second constant domain of the heavy chain), and a CH3 domain (the third constant domain of the heavy chain); and each light chain comprises, from N-terminus to C-terminus, a light chain variable (VL) domain, and a CL domain (the constant domain of the light chain). The anti-CD38 antibody comprises a plurality of linking residues in its constant domains, preferably, in the CH2 and CH3 domains (i.e., CH2-CH3 segments), and the lenalidomide molecules or hydrolyzed lenalidomide molecules are respectively linked to the linking residues of the anti-CD38 antibody. The linking residues are independently selected from the group consisting of K and C residues. According to one embodiment, the anti-CD38 antibody comprises a plurality of K residues in its CH2-CH3 segments; in this case, the lenalidomide molecules or hydrolyzed lenalidomide molecules are respectively linked to the K residues via forming an amide bond therewith. According to another embodiment, the anti-CD38 antibody comprises a plurality of C residues in its CH2-CH3 segments; in this case, the lenalidomide molecules or hydrolyzed lenalidomide molecules are respectively linked to the C residues via reacting with the sulfhydryl (SH) group of the C residues.

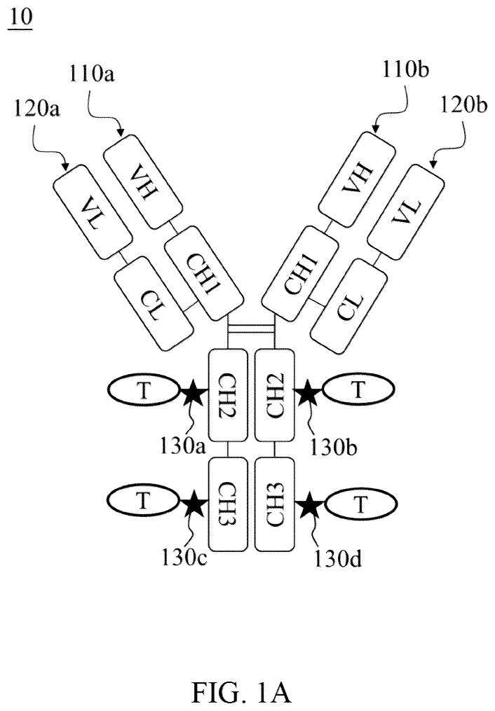

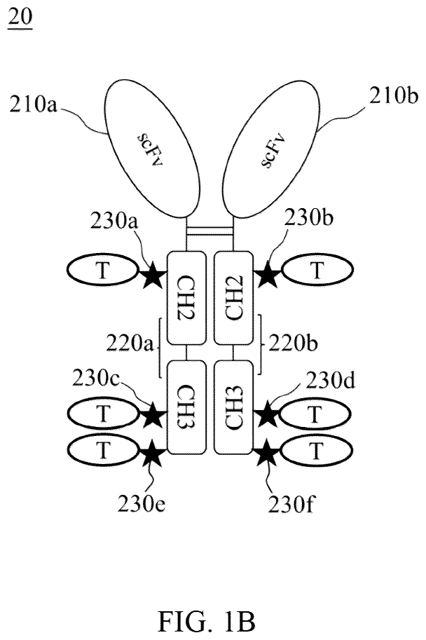

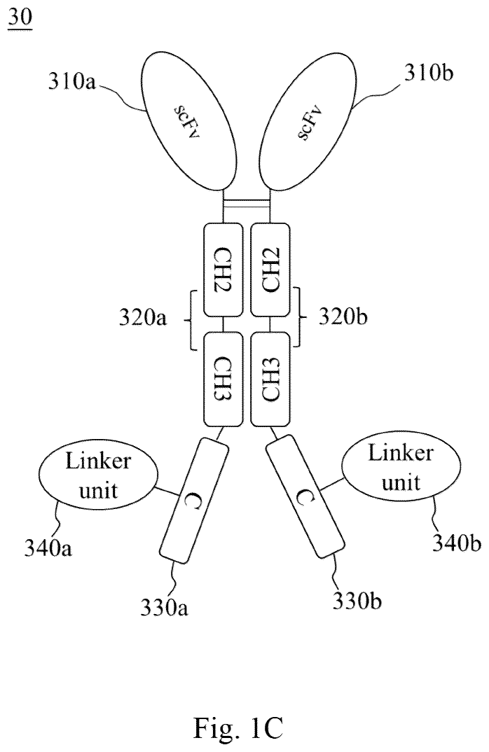

Reference is now made to FIG. 1A, which is a schematic diagram depicting a molecular construct 10 according to some embodiments of the present disclosure. As illustrated, the anti-CD38 antibody of the molecular construct 10 comprises a pair of heavy chains 110a, 110b (each heavy chain 110a, 110b comprising a VH domain, a CH1 domain, a CH2 domain and a CH3 domain) and a pair of light chains 120a, 120b (each light chain 120a, 120b comprising a VL domain and a CL domain) as described above, and four linking residues (the star symbols 130a, 130b, 130c, 130d in FIG. 1A) are respectively disposed in the CH2 and CH3 domains. Accordingly, four therapeutic drugs T (i.e., four lenalidomide molecules or four hydrolyzed lenalidomide molecules) are respectively linked to anti-CD38 antibody via the linking residues 130a, 130b, 130c, 130d.

Depending on intended purpose, each of the lenalidomide molecules and hydrolyzed lenalidomide molecules may be linked to the linking residue in the presence or absence of a linker. Preferably, each of the lenalidomide molecules and hydrolyzed lenalidomide molecules is linked to the linking residue via a linker, for example, a cleavable linker or a non-cleavable linker. Exemplary cleavable linkers include, but are not limited to, protease-sensitive linkers (e.g., valine-citrulline dipeptide, valine-alanine dipeptide, valine-lysine dipeptide, valine-arginine dipeptide and glutamate-valine-citrulline tripeptide), pH-sensitive linkers (e.g., hydrazone linker, ester linker and amide linker), and glutathione-sensitive linkers (e.g., N-Succinimidyl 4-(2-pyridyldithio) butanoate (SPDB) and N-succinimidyl-4-(2-pyridyldithio) pentanoate (SPP)). Non-limiting examples of non-cleavable linker include, malcimidocaproyl (MC), malcimidomethyl cyclohexane-1-carboxylate (MCC), and succinimidyl-4-[N-maleimidomethyl]cyclohexane-1-carboxylate (SMCC). Alternatively, the linker may be a linker known in the art to connect two functional motifs in an immunoconjugate (e.g., connecting the antibody and the payload of an ADC). A skilled artisan may select a suitable linker for producing the present molecular construct in accordance with intended purpose. According to some exemplary embodiments, the linker connecting the present anti-CD38 antibody and lenalidomide molecule/hydrolyzed lenalidomide molecule comprises a polyethylene glycol (PEG) chain and a protease-sensitive linker connected to the PEG chain; preferably, the PEG chain has 1 to 10 repeats of EG units. In one specific embodiment, the linker comprises a PEG chain and a valine-alanine dipeptide connected to the PEG chain, in which the PEG chain has 3 repeats of EG units.

According to some embodiments of the present disclosure, the molecular construct is in a form of an scFv-Fc fusion protein, which comprises an scFv fused to the Fc region of an immunoglobulin (e.g., an IgG). In these embodiments, the anti-CD38 antibody of the molecular construct comprises a pair of CH2-CH3 segments of an immunoglobulin, and a pair of anti-CD38 scFvs respectively linked to the N-termini of the pair of CH2-CH3 segments through a hinge domain. The anti-CD38 antibody comprises a plurality of linking residues in the CH2-CH3 segments, and the lenalidomide molecules or hydrolyzed lenalidomide molecules are respectively linked to the linking residues of the anti-CD38 antibody. The linking residues are independently selected from the group consisting of K and C residues. According to one embodiment, the anti-CD38 antibody comprises a plurality of K residues in its CH2-CH3 segments; in this case, the lenalidomide molecules or hydrolyzed lenalidomide molecules are respectively linked to the K residues via forming an amide bond therewith. According to another embodiment, the anti-CD38 antibody comprises a plurality of C residues in its CH2-CH3 segments; in this case, the lenalidomide molecules or hydrolyzed lenalidomide molecules are respectively linked to the C residues via reacting with the SH group of the C residues. As described above, each lenalidomide molecule/hydrolyzed lenalidomide molecule is preferably linked to the linking residue via a linker, for example, a cleavable linker or a non-cleavable linker.

FIG. 1B provides the schematic diagram of a molecular construct 20 according to some embodiments of the present disclosure. In structure, the anti-CD38 antibody of the molecular construct 20 comprises a pair of CH2-CH3 domains 220a, 220b of an immunoglobulin (e.g., IgG), a pair of anti-CD38 scFvs 210a, 210b respectively linked to N-termini of the pair of CH2-CH3 domains 220a, 220b, and six linking residues (the star symbols 230a, 230b, 230c, 230d, 230c, 230f in FIG. 1B) are respectively disposed in the CH2 and CH3 domains 220a, 220b. Accordingly, six therapeutic drugs T (i.e., six lenalidomide molecules or six hydrolyzed lenalidomide molecules) are respectively linked to anti-CD38 antibody via the linking residues 230a, 230b, 230c, 230d, 230c, 230f.

As could be appreciated, the number of the lenalidomide molecules or hydrolyzed lenalidomide molecules carried by the present molecular construct (in the form of an ADC or an scFv-Fc fusion protein) depends on the number of linking residues comprised in the CH2-CH3 segments of the anti-CD38 antibody. Accordingly, a skilled artisan may adjust the number of the linking residues as necessary to optimize the therapeutic efficacy.

According to alternative embodiments of the present disclosure, the molecular construct is in a form of a drug bundle. In these embodiments, the molecular construct comprises an anti-CD38 antibody and at least one linker unit linked to the anti-CD38 antibody, wherein each linker unit carries a plurality of lenalidomide molecules or hydrolyzed lenalidomide molecules. Specifically, the anti-CD38 antibody comprises a pair of CH2-CH3 segments of an immunoglobulin (e.g., IgG), a pair of anti-CD38 scFvs respectively linked to the N-termini of the pair of CH2-CH3 segments via a hinge domain, and a pair of linking peptides respectively linked to the C-termini of the pair of CH2-CH3 segments. The pair of linking peptides comprises a plurality of C residues, and at least one linker unit is linked to the anti-CD38 antibody via the C residue of the linking peptides. According to some preferred embodiments, each of the pair of linking peptides comprises the amino acid sequence of “CGGHA” (SEQ ID NO: 1), “CPGHA” (SEQ ID NO: 2), “CGAHA” (SEQ ID NO: 3), “CPAHA” (SEQ ID NO: 4), “GCGGHA” (SEQ ID NO: 5), “ACPGHA” (SEQ ID NO: 6), or “GCPGHA” (SEQ ID NO: 7). In one specific embodiment, each of the linking peptides comprises the amino acid sequence of “ACPGHA” (SEQ ID NO: 6). According to certain exemplary embodiments, each of the pair of linking peptides consists of the amino acid sequence of “CGGHA” (SEQ ID NO: 1), “CPGHA” (SEQ ID NO: 2), “CGAHA” (SEQ ID NO: 3), “CPAHA” (SEQ ID NO: 4), “GCGGHA” (SEQ ID NO: 5), “ACPGHA” (SEQ ID NO: 6), or “GCPGHA” (SEQ ID NO: 7). In one exemplary embodiment, each of the linking peptides consists of the amino acid sequence of “ACPGHA” (SEQ ID NO: 6).

Reference is now made to FIG. 1C, which depict the schematic diagram of a molecular construct 30 according to certain embodiments of the present disclosure. As depicted, the anti-CD38 antibody of molecular construct 30 comprises a pair of CH2-CH3 domains 320a, 320b of an immunoglobulin (e.g., IgG), a pair of anti-CD38 scFvs 310a, 310b respectively linked to N-termini of the pair of CH2-CH3 domains 320a, 320b, and a pair of linking peptides 330a, 330b respectively linked to the C-termini of the pair of CH2-CH3 domains 320a, 320b, in which each of the linking peptides 330a, 330b comprises a C residues. In this case, two linker units 340a, 340b are respectively linked to the C residues of the linking peptides 330a, 330b of the anti-CD38 antibody.

As could be appreciated, the number of the linker units linked to the anti-CD38 antibody depends on the number of C residues comprised in the linking peptides. For example, in the case when each of the linking peptides comprises two C residues, then four linker units may be linked to the anti-CD38 antibody. A skilled artisan may adjust the number of the C residues of the linking peptide in accordance with practical uses.

According to some embodiments of the present disclosure, the linker unit comprises a center core and a plurality of linking arms linked to the center core. The center core is a polypeptide that is in a linear form, and comprises 2 to 10 K residues (e.g., 2, 3, 4, 5, 6, 7, 8, 9 or 10 K residues) and a terminal spacer. The linking arms are respectively linked to the K residues of the center core, and the lenalidomide molecules or hydrolyzed lenalidomide molecules are respectively linked to the free-terminus (i.e., the terminus that is not chemically bound to any molecules) of the linking arms.

According to some exemplary embodiments, the molecular construct designated as “native 2-chain (anti-CD38 scFv)-hIgG1.Fc-(Gly)₃-BM-lenalidomide bundle” comprises two linker units respectively conjugated to the pair of linking peptides (each linking peptide having the amino acid sequence of “ACPGHA” (SEQ ID NO: 6; a Zn²⁺-binding motif, and accordingly designated as “BM” in the present disclosure)) of the anti-CD38 antibody, in which each linker unit carries three lenalidomide molecules via the linking arms thus forming a drug bundle at the C-terminus of the anti-CD38 antibody. According to some exemplary embodiments, the molecular construct designated as “stabilized 2-chain (anti-CD38 scFv)-hIgG1.Fc-(Gly)₃-BM-lenalidomide bundle” comprises two linker units respectively conjugated to the pair of linking peptides (each linking peptide having the amino acid sequence of “ACPGHA” (SEQ ID NO: 6)) of the anti-CD38 antibody, in which each linker unit carries three hydrolyzed lenalidomide molecules via the linking arms thus forming a drug bundle at the C-terminus of the anti-CD38 antibody. In one embodiment, the hydrolyzed lenalidomide molecule has the structure of

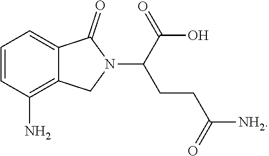 [SMILES: NC(=O)CCC(C(=O)O)N1Cc2c(N)cccc2C1=O]

In another embodiment, the hydrolyzed lenalidomide molecule has the structure of

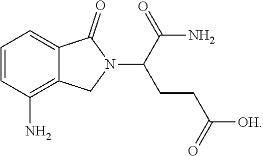 [SMILES: NC(=O)C(CCC(=O)O)N1Cc2c(N)cccc2C1=O]

The linker unit is linked to the C residue of the linking peptide of the anti-CD38 antibody via the terminal spacer. According to various embodiments of the present disclosure, the terminal spacer may be an N-terminal spacer or a C-terminal spacer. In some embodiments, the terminal spacer is an N-terminal spacer having two termini (i.e., a first terminus and a second terminus), in which one of the termini is linked to the N-terminus of the first K residue (starting from the N-terminus of center core) of the center core, and the other of the termini is linked to the C residue of the linking peptide of the anti-CD38 antibody. According to some exemplary embodiments, the N-terminal spacer has a CO₂H group at the first terminus, and a SH-reactive group (e.g., a malcimide, sulfone, haloacetyl, or pyridyl disulfide group) at the second terminus; in this case, the first terminus of the terminal spacer is linked to the N-terminus of the first K residue via forming an amide bond between the CO₂H group of the N-terminal spacer and the NH₂ group of the first K residue; and the second terminus of the terminal spacer is linked to the C residue of the linking peptide of the anti-CD38 antibody via a thiol-maleimide reaction occurred between the SH-reactive group of the C-terminal spacer and the SH group of the C residue.

In some embodiments, the terminal spacer is a C-terminal spacer having two termini (i.e., a first terminus and a second terminus), in which one of the termini is linked to the C-terminus of the last K residue (starting from the N-terminus of center core) of the center core, and the other of the termini is linked to the C residue of the linking peptide of the anti-CD38 antibody. According to some exemplary embodiments, the C-terminal spacer has an NH₂ group at the first terminus and a SH-reactive group (e.g., a maleimide, sulfone, haloacetyl, or pyridyl disulfide group) at the second terminus; in this case, the first terminus of the terminal spacer is linked to the C-terminus of the last K residue via forming an amide bond between the NH₂ group of the C-terminal spacer and the CO₂H group of the last K residue; and the second terminus of the terminal spacer is linked to the C residue of the linking peptide of the anti-CD38 antibody via a thiol-maleimide reaction occurred between the SH-reactive group of the N-terminal spacer and the SH group of the C residue.

According to certain embodiments of the present disclosure, the center core has 3-120 amino acid residues in length, and includes at least 2 lysine (K) residues (e.g., 2, 3, 4, 5, 6, 7, 8, 9, 10, or more K residues) in its amino acid sequence, in which any two of the K residues are adjacent to each other or are separated by a filler.

Each of the filler and the terminal spacer independently comprises, (1) 1 to 12 non-K amino acid residues (e.g., 1, 2, 3, 4, 5, 6, 7, 8, 9, 10, 11 or 12 non-K amino acid residues), or (2) a PEGylated amino acid having 1 to 12 repeats of ethylene glycol (EG) unit (e.g., 1, 2, 3, 4, 5, 6, 7, 8, 9, 10, 11 or 12 repeats of EG units). In general, each of the non-K amino acid residues are independently selected from the group consisting of, glycine (G), aspartic acid (D), glutamic acid (E), serine(S), arginine (R), histidine (H), threonine (T), asparagine (N), glutamine (Q), proline (P), alanine (A), valine (V), isoleucine (I), leucine (L), methionine (M), phenylalanine (F), tyrosine (Y), and tryptophan (W) residues. Preferably, the terminal spacer comprises at least three negative charged amino acid residues at pH=7, such as aspartate (D) and/or glutamate (E) residues. According to some exemplary embodiments, the terminal spacer comprises the amino acid sequence of “EDEDEAGG” (SEQ ID NO: 8), “EGEGEAGG” (SEQ ID NO: 9), or “EGEGE” (SEQ ID NO: 10). In one specific example, the center core comprises the amino acid sequence of “EDEDEGAGGKGAGKGAGKG” (SEQ ID NO: 11).

According to certain embodiments of the present disclosure, the linking arm comprises 2-12 non-K amino acid residues (e.g., 2, 3, 4, 5, 6, 7, 8, 9, 10, 11 or 12 non-K amino acid residues), a PEG chain having 2-24 repeats of EG units (e.g., 2, 3, 4, 5, 6, 7, 8, 9, 10, 11, 12, 13, 14, 15, 16, 17, 18, 19, 20, 21, 22, 23 or 24 EG units), or a combination thereof. In one specific embodiment, the linking arm comprises a PEG chain and a valine-alanine (Val-Ala)dipeptide connected to the PEG chain, in which the PEG chain has 3 repeats of EG units. Optionally, the peptide or PEG chain of the linking arm may be substituted with a polymer of approximately the same length. A polymer comprising carbohydrate or other hydrophilic building blocks is suitable for use as the linking arms.

In structure, each of the linking arms has two termini (i.e., a first terminus and a second terminus), in which the first terminus is linked to one of the K residues of the center core, and the second terminus is linked to the lenalidomide molecule or hydrolyzed lenalidomide molecule. According to some embodiments, the first terminus of the linking arm is linked to the &-amino group of the K residue via forming an amide bond therewith, and the second terminus of the linking arm is linked to the lenalidomide molecule or hydrolyzed lenalidomide molecule via a para-aminobenzyl carbamate (PABC) reagent.

Alternatively, the second terminus of the linking arm has a functional group for linking the lenalidomide molecule or hydrolyzed lenalidomide molecule. Depending on intended purposes, the functional group may be an NH₂, CO₂H, N-hydroxysuccinimidyl (NHS), azide, alkyne, cyclooctyne, tetrazine, or cyclooctene group, and the lenalidomide molecule/hydrolyzed lenalidomide molecule is linked to the second terminus of the linking arm via any of the following chemical reactions,
(1) forming an amide bond therebetween; in this case, the functional group is an NH₂, CO₂H or NHS group, and the lenalidomide molecule/hydrolyzed lenalidomide molecule has an NH₂ or CO₂H group (i.e., the lenalidomide molecule/hydrolyzed lenalidomide molecule is modified with an NH₂ or CO₂H group);
(2) the Copper (I)-catalyzed alkyne-azide cycloaddition reaction (CuAAC reaction), in which one of the functional group and the lenalidomide molecule/hydrolyzed lenalidomide molecule has an azide or a picolyl azide group, whereas the other has an alkyne group;
(3) the inverse electron demand Diels-Alder (iEDDA) reaction, in which one of the functional group and the lenalidomide molecule/hydrolyzed lenalidomide molecule has a tetrazine group, whereas the other has a cyclooctene group (e.g., a TCO or a norbornene group); or
(4) the strained-promoted azide-alkyne click chemistry (SPAAC) reaction, in which one of the functional group and the lenalidomide molecule/hydrolyzed lenalidomide molecule has an azide group, whereas the other has a cyclooctyne group.

According to various embodiments of the present disclosure, the tetrazine group is 1,2,3,4-tetrazine, 1,2,3,5-tetrazine, 1,2,4,5-tetrazine, or derivatives thereof; the cyclooctene group is a norbornene or a trans-cyclooctene (TCO) group; and the cyclooctyne group is selected from the group consisting of, dibenzocyclooctyne (DIBO), difluorinated cyclooctyne (DIFO), bicyclononyne (BCN), and dibenzoazacyclooctyne (DIBAC or DBCO). According to one embodiment of the present disclosure, the tetrazine group is 6-methyl-tetrazine.

As could be appreciated, the number of the lenalidomide molecules or hydrolyzed lenalidomide molecules carried by the present linker unit depends on the number of K residues of the center core (and thus, the number of the linking arms). For example, in the case when the center core comprises three K residues, then three linking arms are respectively linked to the K residues of the center core, and three lenalidomide molecules/hydrolyzed lenalidomide molecules are respectively linked to the free-termini of the linking arms. Alternatively, in the case when the center core comprises ten K residues, then ten linking arms are respectively linked to the K residues of the center core, and ten lenalidomide molecules/hydrolyzed lenalidomide molecules are respectively linked to the free-termini of the linking arms. Accordingly, one of ordinary skill in the art may adjust the number of the lenalidomide molecules or hydrolyzed lenalidomide molecule carried by the linker unit via altering the number of the k residues of the center core (and thus, the number of the linking arms) in accordance with desired purpose.

According to alternative embodiments of the present disclosure, the lenalidomide molecules or hydrolyzed lenalidomide molecules are respectively linked to the anti-CD38 antibody via reacting with the SH group of C residues of the linking peptide via a linker, which, as described above, may be a cleavable linker or a non-cleavable linker.

The tumor treatable with the present method may be a solid tumor or a diffused tumor. Examples of the solid tumor include, but are not limited to, melanomas, esophageal carcinomas, gastric carcinomas, brain tumor, small cell lung cancer, non-small cell lung cancer, bladder cancer, breast cancer, pancreatic cancer, colon cancer, rectal cancer, colorectal cancer, renal cancer, hepatocellular carcinoma, ovary cancer, prostate cancer, thyroid cancer, testis cancer, head and neck squamous cell carcinoma, and a combination thereof. Exemplary diffused tumors include, but are not limited to, ALL, CLL, AML, CML, Hodgkin lymphoma, non-Hodgkin lymphoma (e.g., lymphoplasmacytic lymphoma, follicular lymphoma, diffuse large B-cell lymphoma, mantle cell lymphoma, or marginal zone lymphoma), multiple myeloma, and a combination thereof. According to one embodiment of the present disclosure, the tumor is multiple myeloma.

The subject treatable with the present method is a mammal, for example, a human, mouse, rat, guinea pig, hamster, monkey, swine, dog, cat, horse, sheep, goat, cow, and rabbit. Preferably, the subject is a human.

The molecular construct of the present disclosure may be administered to the subject by an appropriate route, such as oral, enteral, nasal, topical, transmucosal, or parenteral administration. Depending on intended purposes, the parenteral administration may be intratumoral, intramuscular, intravenous, intraperitoneal, or intraarterial injection.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### Example

### Example 1 Synthesis of Mal-Lenalidomide Bundles

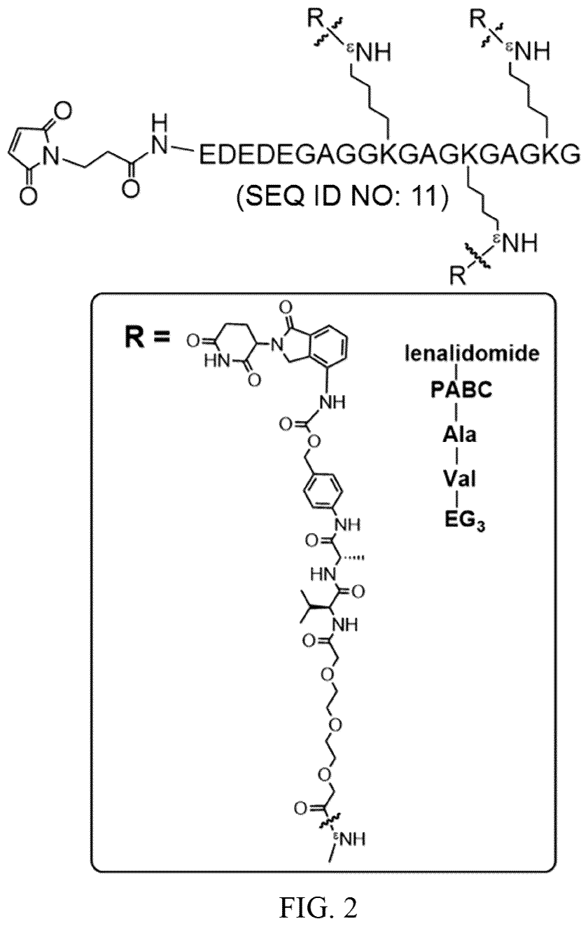

A drug bundle containing three lenalidomide molecules was synthesized in this example. As the structure depicted in FIG. 2, the center core had the sequence of “EDEDEGAGGKGAGKGAGKG” (SEQ ID NO: 11, from N-terminus to C-terminus), and a maleimido-ethyl-CO₂H group (serving as a conjugating group) was linked to the N-terminus of the center core via forming an amide bond between the NH₂ group of the first amino acid residue (i.e., the first “E” residue of SEQ ID NO: 11) and the CO₂H group of the conjugating group. Three linking arms were respectively linked to ε-amino group of the lysine (K) residues of the center core, in which each linking arm comprised 3 repeats of ethylene glycol (EG) unit, a Val-Ala dipeptide that is recognized and cleaved by cathepsin B, and para-aminobenzyl carbamate (PABC) for linking the lenalidomide molecule.

The maleimide-containing lenalidomide bundle (hereinafter as “Mal-lenalidomide bundle”) was synthesized using the combined method, where the standard Fmoc-based solid-phase synthesis was carried out for synthesizing the center core and assembling drug bundle, and the liquid-phase synthesis was conducted for synthesizing the building block. The manufacture was outsourced to WuXi STA Co., Ltd. (Shanghai, China). The synthesis of the present Mal-lenalidomide bundle comprised three steps, including, (i) building block synthesis, (ii) center core synthesis, and (iii) drug bundle assembly. Briefly, Boc-protected Val-Ala dipeptide (compound 1) was sequentially coupled with (4-aminophenyl) methanol (compound 2) and bis(4-nitrophenyl) carbonate in solution to yield compound 3, which in turn was coupled with lenalidomide to give compound 4. After removing the Boc-protecting group, the resulting compound 5 was linked to (EG)₃ via solid-phase peptide synthesis (SPPS) generating compound 6, which was coupled with the ε-amino group of Lys of Fmoc-protected Gly-Lys-Gly tripeptide via SPPS to give building block 7. The center core 8 was synthesized by standard Fmoc-based SPPS and then coupled with 2,3,4,5,6-pentafluorophenol at the C-terminus in solution to yield compound 9. Using building block 7, compound 9, Fmoc-Ala-OH, and N-succinimidyl 3-maleimidopropionate, the lenalidomide bundle 10 was assembled via SPPS.

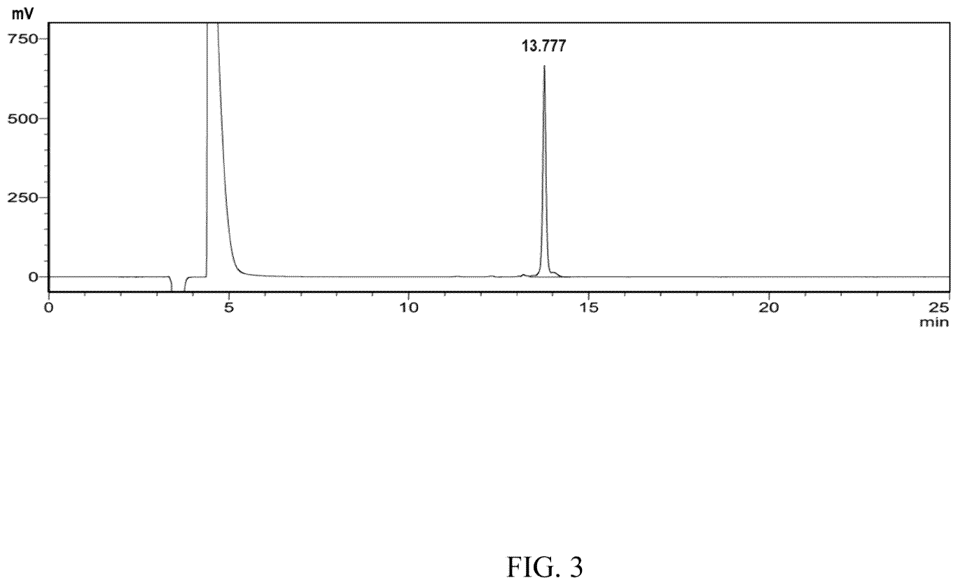

The purified sample of the thus-produced Mal-lenalidomide bundle was analyzed by reversed-phase analytical high-performance liquid chromatography (HPLC). FIG. 3 depicted the reversed-phase HPLC profile of the Mal-lenalidomide bundle, which indicated that the peak of the Mal-lenalidomide bundle had a retention time of 13.777 minutes.

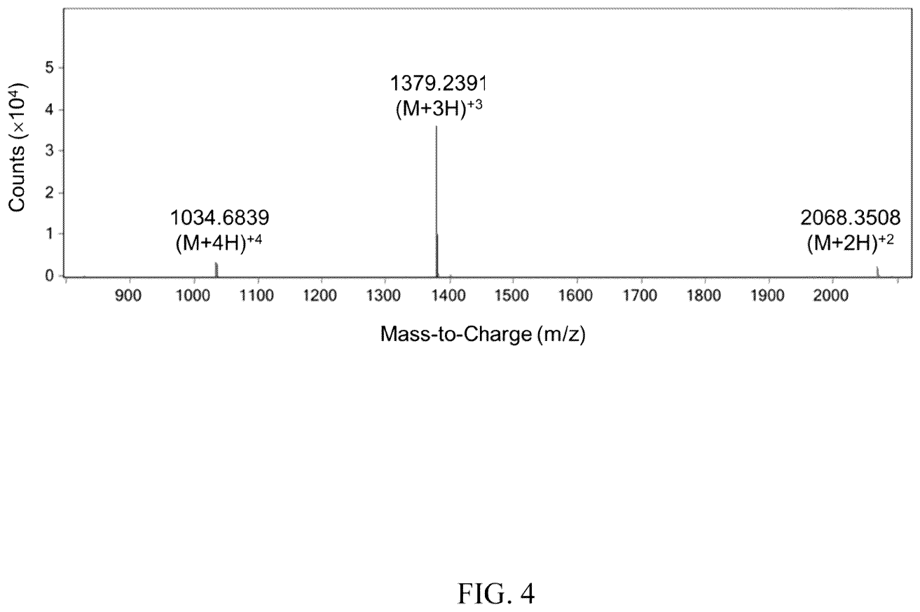

The identification of the Mal-lenalidomide bundle was carried out by mass spectrometry ESI-MS. FIG. 4 depicted the result of mass spectrometry ESI-MS, in which the present molecular construct had a strong molecular ion at 1379.235, which corresponds to [M+3H]³⁺, indicating that the actual molecular weight (M.W.) of Mal-lenalidomide bundle is 1379.235×3−3=4134.705 dalton, in accord with the calculated M.W. of 4135.1474.

### Example 2 Construction of Recombinant 2-Chain (Anti-CD38 scFv)-hIgG1.Fc-(Gly)₃-BM Fusion Protein

The V_{L} and V_{H} of the scFv specific for human CD38 were derived from daratumumab. The gene sequence encoding the anti-CD38 scFv-Fc fusion protein was constructed by fusing a gene sequence encoding the anti-CD38 scFv to the upstream of a gene sequence encoding the Fc region of human IgG1 (hIgG.Fc that comprises a flexible hinge region, CH2 domain and CH3 domain). The gene sequence encoding the Zn²⁺-binding motif, ACPGHA (SEQ ID NO: 6; serving as the linking peptide and designated as “BM” in the present study), was fused to the downstream of the gene sequence encoding the CH3 domain of the hIgG1.Fc (lacking the C-terminal Lys) and a short (Gly) 3 linker. The resulting gene sequence construct was placed in the FREEDOM® pCHO 1.0 expression cassette. The thus-produced fusion protein was in the form of a dimer of (anti-CD38 scFv)-hIgG1.Fc-(Gly)₃-BM, which had the amino acid sequence of SEQ ID NO: 12, in which the anti-CD38 scFvs had the orientation of VL-linker-VH, and the VI, and VH was connected by a hydrophilic linker, GSTSGSGKPGSGEGSTKG (SEQ ID NO: 13).

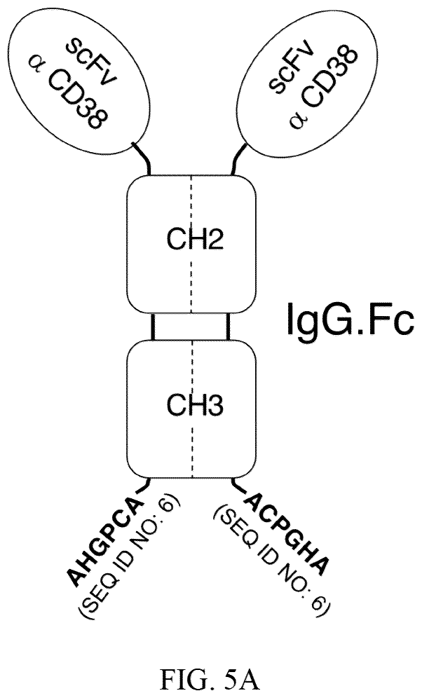

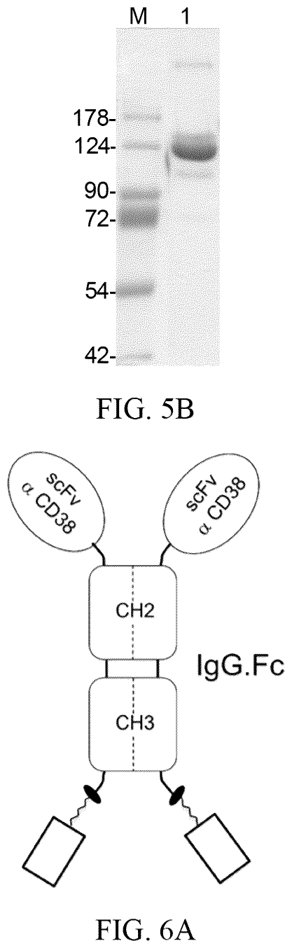

The configuration of the prepared 2-chain (anti-CD38 scFv)-hIgG1.Fc-(Gly)₃-BM fusion protein is provided in FIG. 5A.

### Example 3: Expression and Purification of Recombinant 2-Chain (Anti-CD38 scFv)-hIgG1.Fc-(Gly)₃-BM Fusion Protein

To stably express the fusion protein, the expression plasmid was linearized by SspI digestion and then transfected into CHO-S™ cells. The transfected cells were incubated at 37° C. for 40-48 hours post-transfection in an orbital shaker (150 rpm), followed by incubating in selection medium containing 10 μg/mL puromycin and 200 nM methotrexate (MTX) for stable pool selection. The selection medium was changed twice per week, and the expression of the recombinant 2-chain (anti-CD38 scFv)-hIgG1.Fc-(Gly)₃-BM fusion protein was checked weekly. Two to three weeks later, the cells stably expressing for the 2-chain (anti-CD38 scFv)-hIgG1.Fc-(Gly)₃-BM fusion protein was cryopreserved. The fusion protein was collected from the supernatant of the stable CHO-S™ cells, and purified using protein A chromatography. The buffer was exchanged to phosphate-buffered saline (PBS), and the concentration of the fusion protein was determined and analyzed using SDS-PAGE. The data of FIG. 5B depicted the analytic results, in which the fusion protein was revealed as the major band at about 110 kDa (lane 1 of FIG. 5B), consistent with the expected size. M stands for protein marker. The antibody was dissolved in PBS with 50% glycerol and stored at −20° C. for following study.

### Example 4 Synthesis of the Molecular Construct Containing Recombinant 2-Chain (Anti-CD38 scFv)-hIgG1.Fc-(Gly)₃-BM Fusion Protein and Two Lenalidomide Bundles

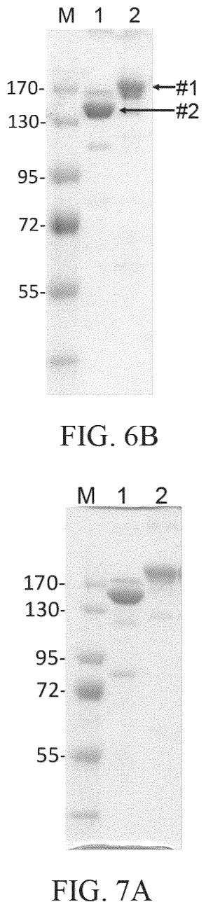

In this example, a molecular construct having two lenalidomide bundles respectively conjugated to the two cysteine residues of the BM (i.e., ACPGHA; SEQ ID NO: 6) of 2-chain (anti-CD38 scFv)-hIgG1.Fc-(Gly)₃-BM was prepared; a schematic diagram illustrating the structure of this molecular construct is provided in FIG. 6A. The purified 2-chain (anti-CD38 scFv)-hIgG1.Fc-(Gly)₃-BM fusion protein of Example 3 was prepared in the conjugation buffer (10 mM sodium succinate, 30 mM sucrose, pH 6.0), in which the final concentration was adjusted to 15 μM; and then reduced by incubating with six equivalents of tris(2-carboxyethyl) phosphine (TCEP) at 37° C. for 30 minutes to free the Zn²⁺-binding Cys, which may form unwanted disulfide bonds with Cys or glutathione in the medium. The reduced protein was dialyzed against the conjugation buffer containing 60 μM ZnCl₂ by slide-A-lyzer dialysis cassette at 25° C. for 2 hours to (i) reconstitute hinge disulfide bonds and (ii) allow the Zn²⁺-binding motif to bind Zn²⁺. Three equivalents of Mal-lenalidomide bundles of Example 1 (45 μM final concentration in 10 mM sodium succinate buffer, 30 mM sucrose, 60 μM ZnCl₂, pH 6.0) were added and incubated with the fusion proteins in the round-bottomed flask under stirring (600 rpm) at 25° C. for 10 minutes. To solubilize the products, an equal volume of 100% (w/v) sucrose was added to the resulting solution and stirred (600 rpm) at 25° C. for 16 to 18 hours. The conjugation product was analyzed by SDS-PAGE.

As the data depicted in FIG. 6B, the molecular construct comprising two lenalidomide bundles had a M.W. of about 120 kDa (see, the protein band labeled as “#1” in lane 2), which was somewhat larger than the expected size. The unconjugated molecular construct was in lane 1 and labeled as #2 in FIG. 6B. M stands for protein marker. As depicted in FIG. 6B, the yield of the conjugation of the 2-chain (anti-CD38 scFv)-hIgG1.Fc-(Gly)₃-BM fusion protein with two lenalidomide bundles (hereinafter as “native 2-chain (anti-CD38 scFv)-hIgG1.Fc-(Gly)₃-BM-lenalidomide bundle”) was approximately 85%.

### Example 5 Stabilization of Native 2-Chain (Anti-CD38 scFv)-hIgG1.Fc-(Gly)₃-BM-Lenalidomide Bundles in the Basic Solution

To prevent β-elimination reaction of the thioether bond, the thiol-maleimide conjugates were hydrolyzed to induce maleimide ring opening by adding 1/10 volume of the hydrolysis buffer (100 mM Tris, 100 mM NaCl, 100 mM L-Arginine and 50% sucrose (w/v), pH 9.0) to the 2-chain (anti-CD38 scFv)-hIgG1.Fc-(Gly)₃-BM-lenalidomide bundles of Example 4. The resulting solution was then stirred at 25° C. over 2 days to hydrolyze the maleimide ring.

The reaction product of the molecular construct obtained in Example 4 was buffer-exchanged into a Tris buffer (100 mM Tris at pH 9.0, 100 mM L-Arginine, and 100 mM sodium chloride) using a column. The resulting solution was then heated to 37° C. for 5 hours. The solution was cooled and buffer-exchanged by centrifugation into 50 mM Bis-Tris buffer at pH 5.5. Final samples were concentrated to about 1 to 3 mg/mL protein. The thus-produced product was designated as “stabilized 2-chain (anti-CD38 scFv)-hIgG1.Fc-(Gly)₃-BM-lenalidomide bundles”.

### Example 6 Purification of the Stabilized 2-Chain (Anti-CD38 scFv)-hIgG1.Fc-(Gly)₃-BM-Lenalidomide Bundles

Before purification, the stabilized product of Example 5 was exchanged to buffer A (50 mM Na₂HPO₄, 1 M NaCl, pH 7.0). To remove unconjugated molecules or conjugates with only one drug bundle, the mixture was applied to pre-equilibrated hydrophobic interaction column (HIC), followed by three washing steps with buffer B (50 mM sodium phosphate, pH 7.0) of 0, 50, and 65% for 10, 20 and 15 column volumes, respectively. The conjugates with 2 drug bundles were eluted with 100% buffer B for 20 column volumes at a flow rate of 1.0 ml/min. The collected samples of the preceding HIC purification was applied to a size exclusion chromatography (SEC) column to separate the molecular construct from protein aggregates. After HIC and SEC purification, the stabilized 2-chain (anti-CD38 scFv)-hIgG1.Fc-(Gly)₃-BM-lenalidomide bundle was acquired.

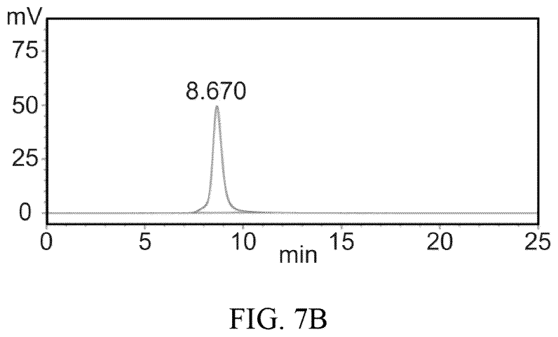

FIG. 7A depicted the results of SDS-PAGE analysis of the stabilized 2-chain (anti-CD38 scFv)-hIgG1.Fc-(Gly)₃-BM-lenalidomide bundles, in which the lane labeled with 1 and the lane labeled with 2 respectively corresponded to unconjugated molecular construct and molecular construct conjugated with two lenalidomide bundles. The result of elution profile of size-exclusive chromatography indicated that the stabilized 2-chain (anti-CD38 scFv)-hIgG1.Fc-(Gly)₃-BM-lenalidomide bundles had a purity of >95% (FIG. 7B).

### Example 7 Purification of the Native 2-Chain (Anti-CD38 scFv)-hIgG1.Fc-(Gly)₃-BM-Lenalidomide Bundles

To obtain the native 2-chain (anti-CD38 scFv)-hIgG1.Fc-(Gly)₃-BM-lenalidomide bundles, the product of Example 4 was exchanged to buffer A (50 mM Na₂HPO₄, 1 M NaCl, pH 6.0) prior to purification. Briefly, the reaction mixture was applied to pre-equilibrated hydrophobic interaction column (HIC), followed by three washing steps with buffer B (50 mM sodium phosphate, pH 6.0) of 0, 50, and 65% for 10, 20 and 15 column volumes, respectively. The conjugates with 2 lenalidomide bundles were eluted with 100% buffer B for 20 column volumes at a flow rate of 1.0 ml/min. The collected samples of the preceding HIC purification were applied to a size exclusion chromatography column to separate the molecular construct from protein aggregates. After HIC and SEC purification, the native 2-chain (anti-CD38 scFv)-hIgG1.Fc-(Gly)₃-BM-lenalidomide bundle was acquired in high purity.

### Example 8 Establishment of HPLC-Based Lenalidomide Releasing Assay

To evaluate the release of free lenalidomide from the stabilized 2-chain (anti-CD38 scFv)-hIgG1.Fc-(Gly)₃-BM-lenalidomide bundles after internalization, the drug releasing assay was established. Briefly, lenalidomide (1 μM) dissolved in PBS or RPMI medium, the stabilized 2-chain (anti-CD38 scFv)-hIgG1.Fc-(Gly)₃-BM-lenalidomide bundles (1 μM) dissolved in RPMI medium, H929 cells cultured in RPMI medium, and the stabilized 2-chain (anti-CD38 scFv)-hIgG1.Fc-(Gly)₃-BM-lenalidomide bundles (1 μM) co-cultured with 1×10⁶ H929 cells in RPMI medium were respectively incubated at 37° C. for 5 days. All the supernatants were collected from above mentioned conditions and centrifuged to remove cell debris. Molecules with M.W. lower than 3 kDa were collected by columns. The prepared samples were injected into the C8 column for HPLC analysis. The HPLC system was operated in gradient mode with a flow rate of 1 mL/min. Solvent A consisted of water with 0.1% trifluoroacetic acid (TFA), and solvent B consisted of acetonitrile with 0.1% TFA. The HPLC program, which started at 0% solvent B, was maintained for 8 minutes, then a linear gradient started at 0% solvent B and increased to 100% solvent B within 20 minutes. After pure solvent B was maintained for 10 minutes, the column was regenerated with 100% solvent A within 7 minutes. The injection volume was 100 mL, and the column oven temperature was set at 25° C. The total run time was 45 minutes.

### Example 9 Analyzing the Structures of the Lenalidomide Molecules Released from the Stabilized 2-Chain (Anti-CD38 scFv)-hIgG1.Fc-(Gly)₃-BM-Lenalidomide Bundles

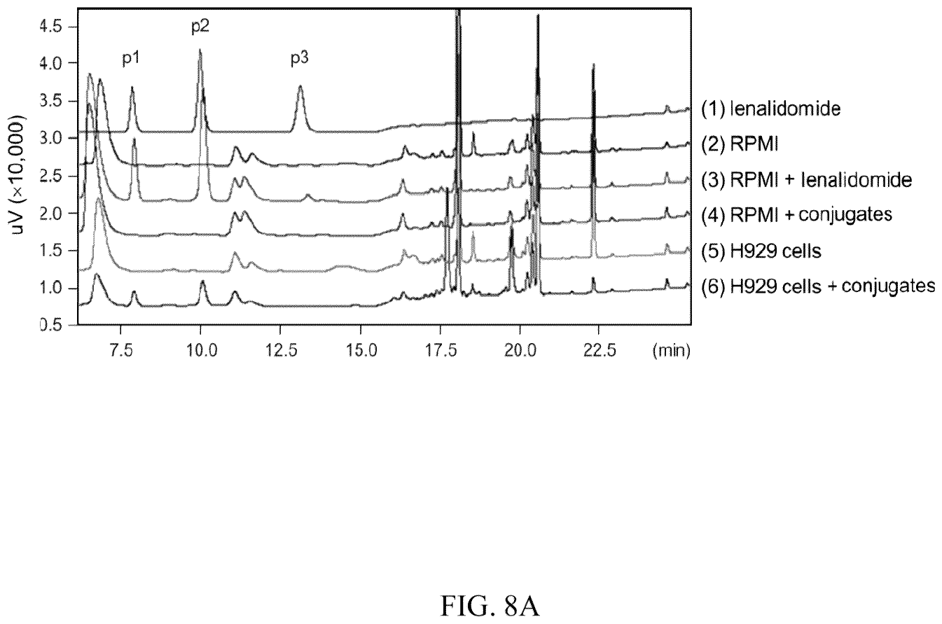

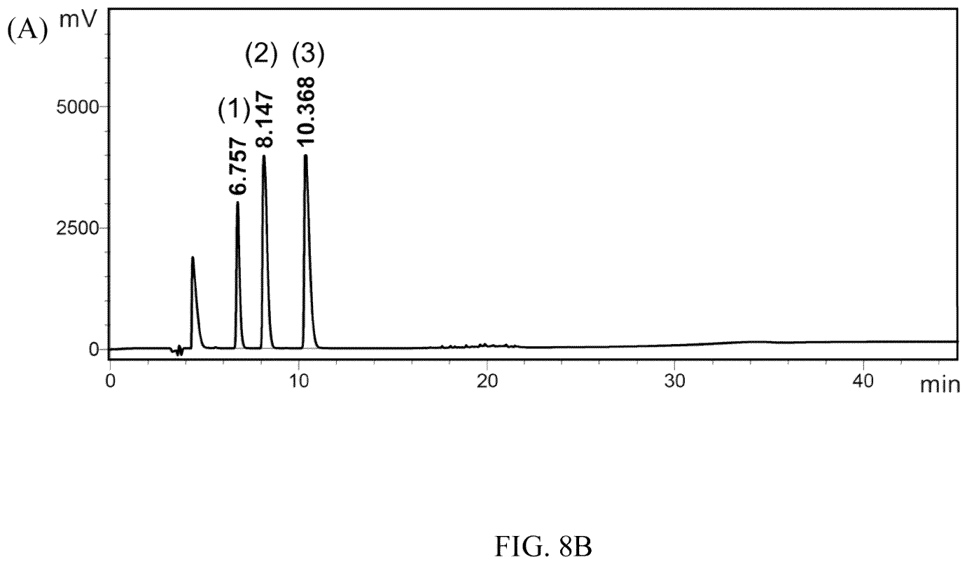

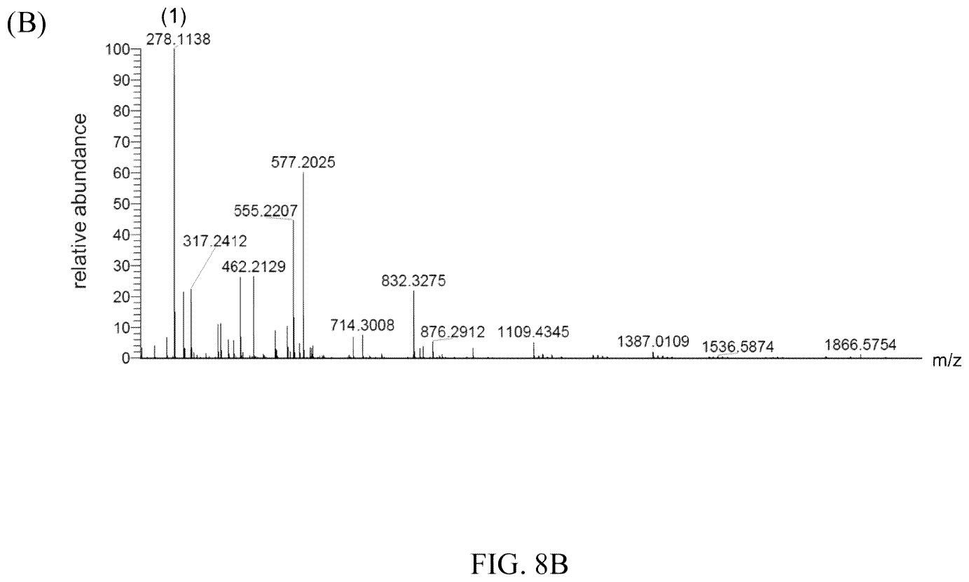

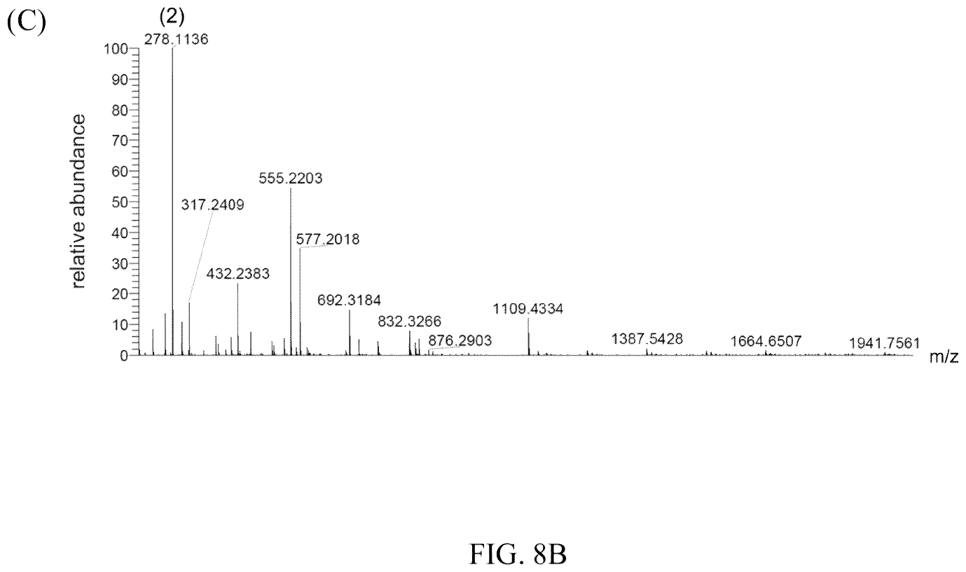

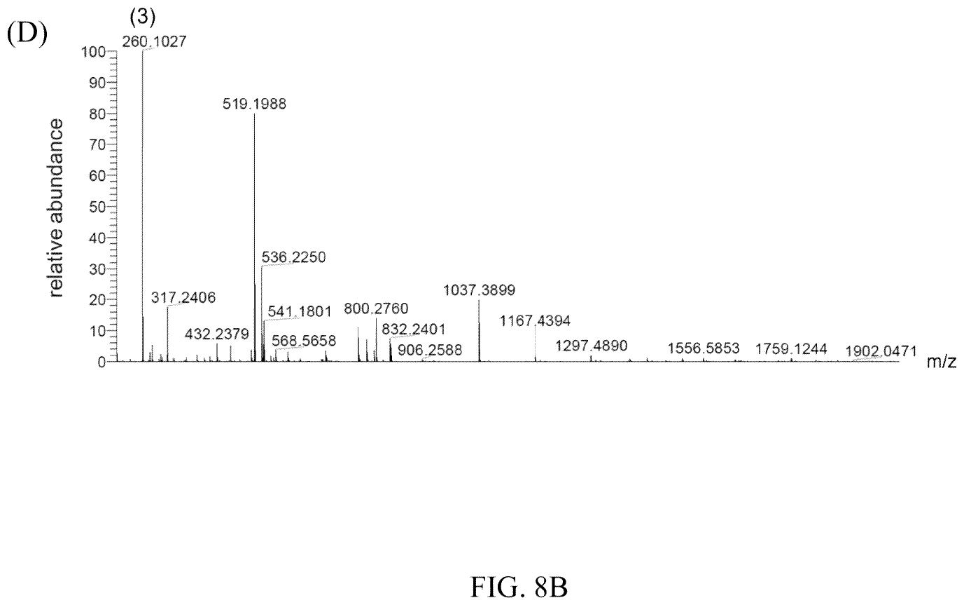

In the study of analyzing the structures of released lenalidomide molecules, lenalidomide incubated in PBS for 5 days served as a control group, which exhibited a peak (referred to as p3) corresponding to the lenalidomide molecule, and two additional peaks (p2 and p1) respectively corresponding to its two hydrolyzed forms after long-term incubation (FIG. 8A, group (1): lenalidomide). The three peaks in the HPLC profiles were confirmed by mass spectrometry. According to the analytic results as depicted in FIG. 8B, three peaks (respectively referred to as (1), (2) and (3)) were detected after replacing lenalidomide in PBS with RPMI medium, in which peak (1) corresponded to the hydrolyzed lenalidomide having the structure of

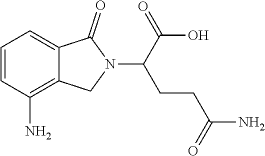 [SMILES: NC(=O)CCC(C(=O)O)N1Cc2c(N)cccc2C1=O]

(Panels (A) and (B) of FIG. 8B); peak (2) corresponded to the hydrolyzed lenalidomide having the structure of

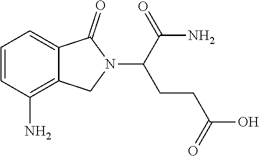 [SMILES: NC(=O)C(CCC(=O)O)N1Cc2c(N)cccc2C1=O]

(Panels (A) and (C) of FIG. 8B); and peak (3) corresponded to the lenalidomide having the structure of

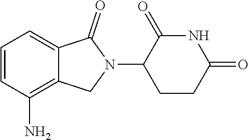 [SMILES: Nc1cccc2c1CN(C1CCC(=O)NC1=O)C2=O]

(Panels (A) and (D) of FIG. 8B). Two hydrolyzed forms of lenalidomide (p1 and p2) could be detected in culture supernatants of H929 cells incubated with the stabilized 2-chain (anti-CD38 scFv)-hIgG1.Fc-(Gly)₃-BM-lenalidomide bundles for 5 days, and no original lenalidomide (p3) was detected (FIG. 8A, group (6): H929 cells+conjugates). No p1, p2, or p3 peaks were found in RPMI medium alone (FIG. 8A, group (2): RPMI), the stabilized 2-chain (anti-CD38 scFv)-hIgG1.Fc-(Gly)₃-BM-lenalidomide bundles incubated in RPMI medium for 5 days (FIG. 8A, group (4): RPMI+conjugates), or the supernatant of H929 cells cultured in RPMI medium for 5 days (FIG. 8A, group (5): H929 cells).

Therefore, these results indicated that the lenalidomide molecules carried by the stabilized 2-chain (anti-CD38 scFv)-hIgG1.Fc-(Gly)₃-BM fusion protein were released in the hydrolyzed forms (p1 and p2).

### Example 10 MALDI-TOF Analysis of Stabilized 2-Chain (Anti-CD38 scFv)-hIgG1.Fc-(Gly)₃-BM-Lenalidomide Bundles for Determination of DAR

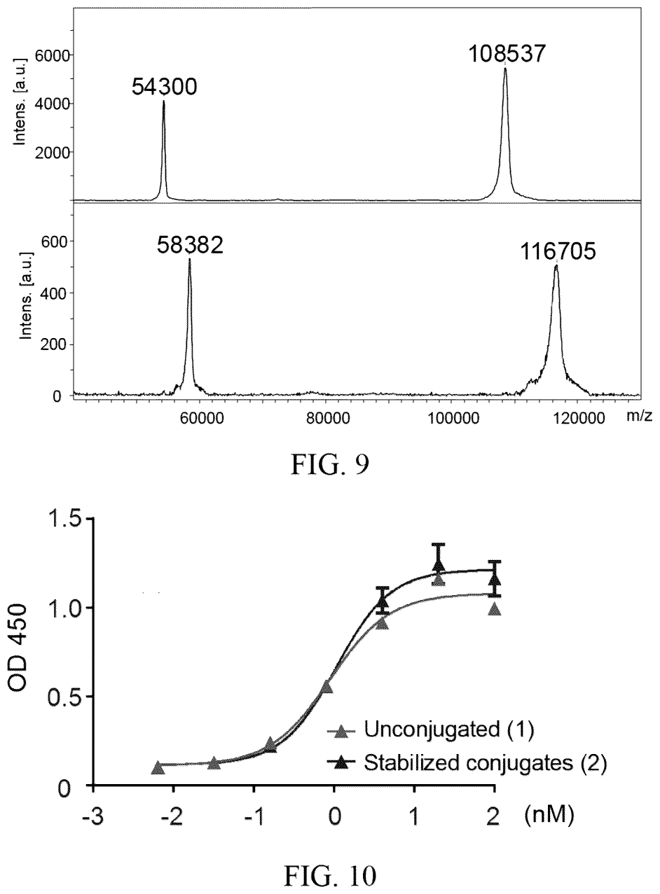

The stabilized 2-chain (anti-CD38 scFv)-hIgG1.Fc-(Gly)₃-BM-lenalidomide bundle was analyzed by matrix-assisted laser desorption/ionization time-of-flight (MALDI-TOF). It was directly spotted onto a MALDI target plate using saturated sinapinic acid as the desorption matrix (2 mg/mL in 0.1% TFA in 30:70 acetonitrile: water, v/v). Each spot was analyzed on a MALDI TOF/TOF equipped with a 200 Hz laser. Data acquisition and processing were made by software. The analytic results indicated that the stabilized 2-chain (anti-CD38 scFv)-hIgG1.Fc-(Gly)₃-BM-lenalidomide bundles had a M.W. of 58,382 and 116,705 daltons, respectively corresponding to m/z (z=1): [M+H]⁺ and m/z (z=2): [M+2H]²⁺ (the lower panel of FIG. 9); and the 2-chain (anti-CD38 scFv)-hIgG1.Fc-(Gly)₃-BM fusion protein (drug free, serving as a control) had a M.W. of 54,300 and 108,537 daltons, which correspond to m/z (2=1): [M+H]⁺ and m/z (2=2): [M+2H]²⁺ (the upper panel of FIG. 9).

In MALDI-TOF analysis, the observed m/z values of the singly (Z=1) and doubly (Z=2) charged stabilized 2-chain (anti-CD38 scFv)-hIgG1.Fc-(Gly)₃-BM-lenalidomide bundles were 116,820 and 58,501 Da, respectively; and the corresponding m/z values for the drug-free 2-chain (anti-CD38 scFv)-hIgG1.Fc-(Gly)₃-BM fusion protein were 108,534 and 54,311 Da, respectively (data not shown). As the lenalidomide bundle had a M.W. of 4,205 Da, the M.W. increase of 8,286 Da of the stabilized 2-chain (anti-CD38 scFv)-hIgG1.Fc-(Gly)₃-BM-lenalidomide bundles over unconjugated 2-chain (anti-CD38 scFv)-hIgG1.Fc-(Gly)₃-BM fusion protein for the Z=1 species represented the M.W. of two lenalidomide bundles. Hence, the stabilized 2-chain (anti-CD38 scFv)-hIgG1.Fc-(Gly)₃-BM-lenalidomide bundles molecule has a drug-antibody ratio (DAR) of 6.

### Example 11 Analyzing Drug Bundle Conjugation Site on the Stabilized 2-Chain (Anti-CD38 scFv)-hIgG1.Fc-(Gly)₃-BM-Lenalidomide Bundles

In order to identify the cysteine residues in the stabilized 2-chain (anti-CD38 scFv)-Fc-BM-lenalidomide bundles, the sample was digested and analyzed using LC-MS. Briefly, to reduce the disulfide bonds, the stabilized 2-chain (anti-CD38 scFv)-hIgG1.Fc-(Gly)₃-BM-lenalidomide bundles (2 μg) was dissolved in PBS at pH 7.4 and incubated with 18 mM TCEP in 17 mM triethylammonium bicarbonate (TEAB) buffer at 55° C. for 10 minutes away from light. After alkylation with iodoacetamide (IAA) at a final concentration of 15.6 mM in the dark at room temperature for 30 minutes, each analyte was incubated with trypsin (0.2 μg) at 37° C. overnight. The samples were detected by liquid chromatography (LC) ESI-MS on mass spectrometer. The digested solution (5 μl) was injected into a capillary column (C18, 0.075 mm×150 mm, ID 3 μm) at a flow rate of 1 μl/min. The following gradient program was used for chromatographic separation: A flow rate of 300 nL/min using initially 100% mobile phase A (0.1% formic acid in water), which was reduced to 98% by 2% mobile phase B (0.1% formic acid in 80% acetonitrile) at 2 minutes and further reduced to 60% by 40% mobile phase B at 40 minutes. Full-scan mass spectrometry was recorded, and the target m/z was isolated for collision-induced dissociation with NCE35 and a maximum injection time of 100 ms. The LC-MS spectra were searched for expected molecular weights of peptides (1366 daltons) with an additional M.W. of a carbamidomethyl group (58 daltons) or a lenalidomide bundle (4205 daltons). The Mascot search engine was used to identify cysteine-containing peptide sequences.

The mass spectrometric analysis result indicated that the m/z value of the fragment in the MS spectrum corresponded to 5571.29 daltons (data not shown), which matched the molecular weight of the fragment containing the amino acid sequence of “SLSLSPGGGGACPGHA” (SEQ ID NO: 14; amino acid residues 472-487 of SEQ ID NO:12) (1,366 daltons) of the molecular construct and one lenalidomide bundle (4,205 daltons).

### Example 12 Generating Stable Human CD38-Overexpressing HEK293T Cells

The cDNA of full-length human CD38 was cloned into pCDH-CMV-MCS-Ef1α-Puro expression vector to generate lentiviral-based CD38-expressing plasmid, pCDH-CMV-CD38. Lentivirus was packaged by co-transfecting pCDH-CMV-CD38, pCMV-VSV-G, and pCMV8R8.91 into HEK293T cells. Human CD38-overexpressing HEK293T cells were transduced with lentivirus infection and cultured with 1 μg/mL puromycin three days post-infection. The expression of human CD38 was confirmed by flow cytometry.

### Example 13 Binding Activity of Stabilized 2-Chain (Anti-CD38 scFv)-hIgG1.Fc-(Gly)₃-BM-Lenalidomide Bundles Toward Human CD38-Overexpressing HEK293T Cells

To ensure that the two lenalidomide bundles conjugated at the antibody C-termini did not alter the antibody's targeting ability, the binding activities of the 2-chain (anti-CD38 scFv)-hIgG1.Fc-(Gly)₃-BM fusion protein and the stabilized 2-chain (anti-CD38 scFv)-hIgG1.Fc-(Gly)₃-BM-lenalidomide bundles to human CD38 were determined by cell-based ELISA using human CD38-overexpressing 293T cells. The stabilized 2-chain (anti-CD38 scFv)-hIgG1.Fc-(Gly)₃-BM-lenalidomide bundles (labeled as “stabilized conjugates”) and the 2-chain (anti-CD38 scFv)-hIgG1.Fc-(Gly)₃-BM fusion protein (labeled as “unconjugated”) exhibited similar binding activity to human CD38 (FIG. 10). According to the results, the EC₅₀ values of the 2-chain (anti-CD38 scFv)-hIgG1.Fc-(Gly)₃-BM-lenalidomide bundles and the 2-chain (anti-CD38 scFv)-hIgG1.Fc-(Gly)₃-BM fusion protein were 0.65 and 0.84 nM, respectively. Hence, the conjugation of two lenalidomide bundles to the 2-chain (anti-CD38 scFv)-hIgG1.Fc-(Gly)₃-BM fusion protein did not compromise binding affinity for human CD38.

### Example 14 Binding Activity of Stabilized 2-Chain (Anti-CD38 scFv)-hIgG1.Fc-(Gly)₃-BM-Lenalidomide Bundle Toward Human Multiple Myeloma H929 Cell Line

The binding affinity of the stabilized 2-chain (anti-CD38 scFv)-hIgG1.Fc-(Gly)₃-BM-lenalidomide bundles to live human CD38⁺ multiple myeloma cells (including H929 and U266-CD38⁺ cells) was examined in this example.

Briefly, about 2×10⁵ of H929 cells or U266-CD38⁺ cells were washed with flow cytometry staining (FACS) buffer (PBS with 1% fetal bovine serum) thrice and incubated with different concentrations (1 μM, 100 nM, 10 nM, 1 nM, 100 pM, 10 PM and 1 pM) of the stabilized 2-chain (anti-CD38 scFv)-hIgG1.Fc-(Gly)₃-BM-lenalidomide bundles and daratumumab for 20 minutes. After washing using FACS buffer, cells were labeled with phycoerythrin (PE)-conjugated anti-human IgG-Fc antibody to detect the level of the stabilized 2-chain (anti-CD38 scFv)-hIgG1.Fc-(Gly)₃-BM-lenalidomide bundles and daratumumab on the cell surface. Cells without staining and cells treated with secondary antibodies only were used as negative controls. The cell-associated fluorescence was determined using flow cytometry and analyzed by software.

Like the “parent” daratumumab (labeled as “dara”), the stabilized 2-chain (anti-CD38 scFv)-hIgG1.Fc-(Gly)₃-BM-lenalidomide bundles (labeled as “stabilized conjugates”) exhibited binding activity to H929 and U266-CD38+ cells in a specific and dose-dependent manner (FIG. 11). However, the EC₅₀ values for the stabilized 2-chain (anti-CD38 scFv)-hIgG1.Fc-(Gly)₃-BM-lenalidomide bundles to H929 cells (4.06 nM) and U266-CD38+ cells (1.66 nM) were greater than those for the “parent” daratumumab (1.02 and 0.13 nM, respectively), indicating that the binding activity of the stabilized 2-chain (anti-CD38 scFv)-hIgG1.Fc-(Gly)₃-BM-lenalidomide bundles to human CD38 was lower than that of daratumumab.

### Example 15 Cellular Internalization of Stabilized 2-Chain (Anti-CD38 scFv)-hIgG1.Fc-(Gly)₃-BM-Lenalidomide Bundles

The internalization assay was performed in the example to examine the extent of internalization of the stabilized 2-chain (anti-CD38 scFv)-hIgG1.Fc-(Gly)₃-BM-lenalidomide bundles into H929 cells.

In brief, 2×10⁵/well H929 cells were incubated with 1 μM stabilized 2-chain (anti-CD38 scFv)-hIgG1.Fc-(Gly)₃-BM-lenalidomide bundles (in FACS buffer) for 20 minutes on ice. After washing and resuspending by fresh medium, the cells were incubated at 37° C. for 0.5, 1, 2, or 3 hours. The cells were washed and incubated with PE-conjugated goat anti-human IgG-Fc antibody for 20 minutes on ice. After washing, the cell-associated fluorescence was determined using flow cytometry and analyzed by software. The internalization percentage was calculated from dM, the mean fluorescence intensity at a given time t relative to the background mean fluorescence intensity, according to equation (1):

$$\% internalization = [ dM ( t = 0 ) - dM ( t = x ) ] \times 100 / dM ( t = 0 ) \qquad ( 1 )$$

The signal intensity of the stabilized 2-chain (anti-CD38 scFv)-hIgG1.Fc-(Gly)₃-BM-lenalidomide bundles of live H929 cells decreased by about 15% after 30 minutes of incubation and by another 60% after 3 hours (Table 1, live cells), indicating that fewer molecules of the stabilized 2-chain (anti-CD38 scFv)-hIgG1.Fc-(Gly)₃-BM-lenalidomide bundles remained bound on the cell surface. In contrast, no loss of signal intensity of the stabilized 2-chain (anti-CD38 scFv)-hIgG1.Fc-(Gly)₃-BM-lenalidomide bundles of fixed cells was detected after 3 hours of incubation (Table 1, fixed cells), indicating that the reduction of signal intensity in live cells did not stem from the dissociation of molecular construct during incubation.

TABLE 1
The kinetics of internalization of the stabilized 2-chain (anti-
CD38 scFv)-hIgG1.Fc-(Gly)₃-BM-lenalidomide bundles in H929 cells

| | |
|---|---|
| Time of incubation (hour) | Signal intensity (PE) |

Live cells

| | |
|---|---|
| 0 | 84.5% |
| 0.5 | 68.0% |
| 1 | 19.2% |
| 2 | 11.8% |
| 3 | 8.89% |

Fixed cells

| | |
|---|---|
| 0 | 67.0% |
| 3 | 65.9% |

### Example 16 In Vitro Cytotoxic Activities of Stabilized 2-Chain (Anti-CD38 scFv)-hIgG1.Fc-(Gly)₃-BM-Lenalidomide Bundles Through the Hydrolyzed Lenalidomide Molecules Releasing

To assess the in vitro cytotoxic activity of the stabilized 2-chain (anti-CD38 scFv)-hIgG1.Fc-(Gly)₃-BM-lenalidomide bundles, alamarBlue™ cell viability assays were carried out. Briefly, H929, U266-CD38⁻, MM.1S, and Daudi cells were seeded at 5,000 cells/well in 96-well plates and co-cultured with fresh medium containing different test articles with the indicated concentrations at 37° C. for 5 hours, 1 day, 3 days, or 5 days. The viabilities of the cells were determined by alamarBlue™ cell viability reagent. About 10 μl alamarBlue™ cell viability reagent was added to the cells with a final concentration of 10% v/v, followed by incubating at 37° C. for 1.5 hours. The fluorescence was measured on a microplate reader in arbitrary fluorescent units following excitation at 560 nm and emission at 590 nm.

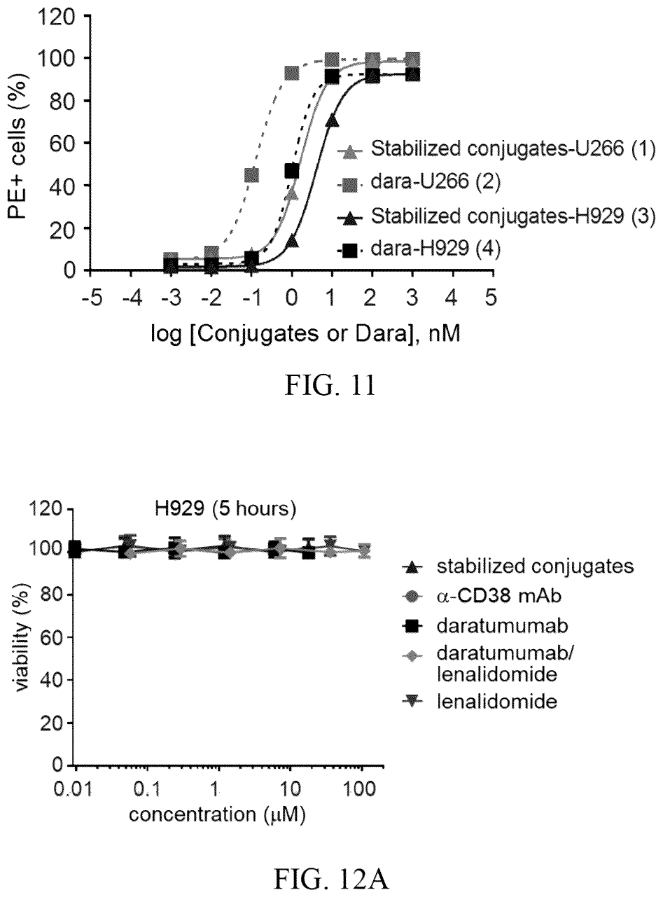

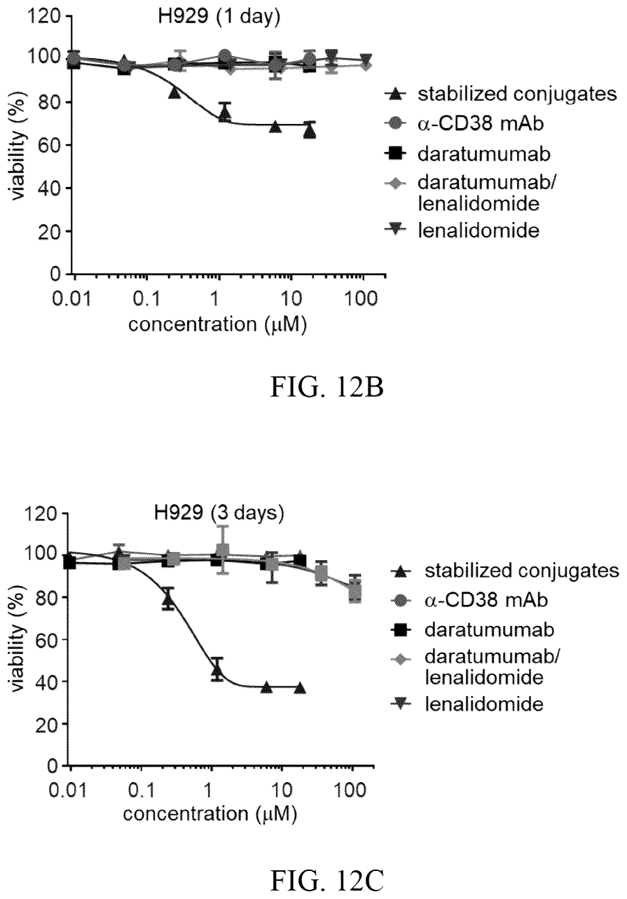

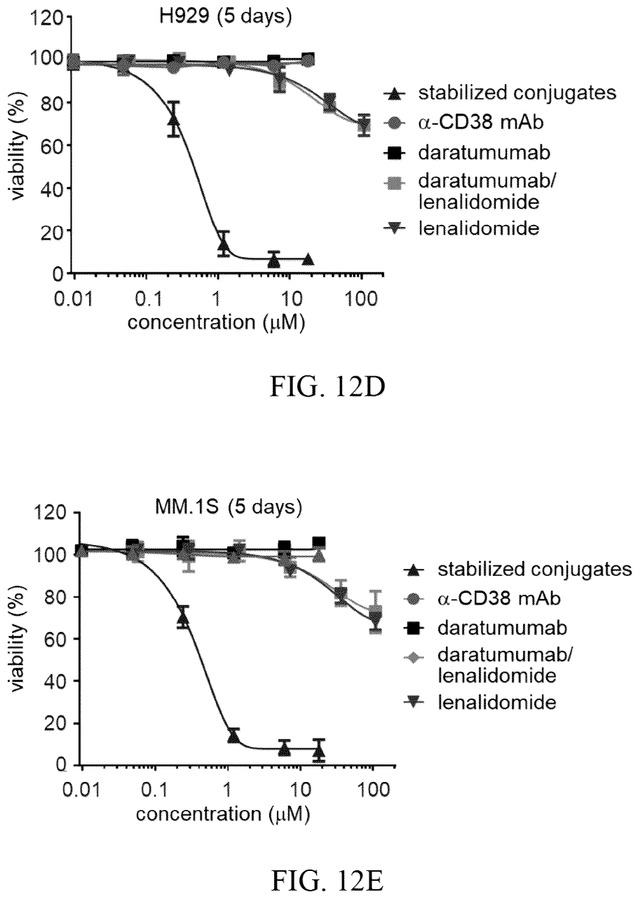

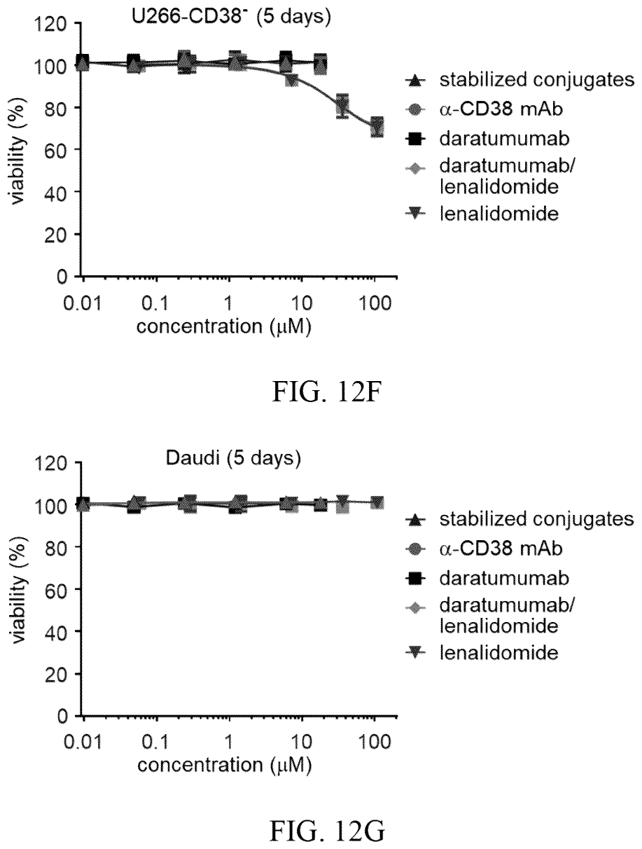

The in vitro tumor cell killing effects of the stabilized 2-chain (anti-CD38 scFv)-hIgG1.Fc-(Gly)₃-BM-lenalidomide bundles on a panel of multiple myeloma (MM) cells was compared with three other anti-MM drugs, including (1) daratumumab, (2) daratumumab combined with lenalidomide at a 1:6 molar ratio that was analogous to the 2-chain (anti-CD38 scFv)-hIgG1.Fc-(Gly)₃-BM fusion protein combined with lenalidomide in the stabilized 2-chain (anti-CD38 scFv)-hIgG1.Fc-(Gly)₃-BM-lenalidomide bundles, and (3) lenalidomide (equal to the amount of lenalidomide used in the daratumumab/lenalidomide combination). Different MM cells were incubated with serial dilutions of the stabilized 2-chain (anti-CD38 scFv)-hIgG1.Fc-(Gly)₃-BM-lenalidomide bundles (labeled as “stabilized conjugates”), the 2-chain (anti-CD38 scFv)-hIgG1.Fc-(Gly)₃-BM fusion protein (unconjugated, labeled as “α-CD38 mAb”), daratumumab, daratumumab and lenalidomide combination (labeled as “daratumumab/lenalidomide”) and lenalidomide. H929 cells were incubated for 5 hours, 1 day, 3 days, or 5 days (FIGS. 12A-12D), and MM.1S, U266-CD38-, and Daudi cells were incubated for 5 days (FIGS. 12E-12G). Then, cell viability was determined.

The treatment of the stabilized 2-chain (anti-CD38 scFv)-hIgG1.Fc-(Gly)₃-BM-lenalidomide bundles (i.e., “stabilized conjugates”) exhibited a dose-dependent cytotoxic effect with an EC₅₀ of ˜0.45 μM on both H929 and MM.1S cells (FIGS. 12B-12E). In contrast, the 2-chain (anti-CD38 scFv)-hIgG1.Fc-(Gly)₃-BM fusion protein (unconjugated, i.e., “anti-CD38 mAb”) and parental daratumumab exhibited no effect of cell death on the same cells even at doses >20 μM, as daratumumab-mediated cell death depends on the presence of other immune cells such as natural killer cells (FIGS. 12B-12E). Lenalidomide or daratumumab/lenalidomide combination at doses >10 μM exhibited only weak killing of H929 and MM.1S cells (FIGS. 12B-12E), indicating that only a tiny fraction of lenalidomide gets into target MM cells, and once inside, it exhibited cytotoxic effect on MM cells. Although hydrolyzed lenalidomide (rather than lenalidomide) molecules were released from the stabilized 2-chain (anti-CD38 scFv)-hIgG1.Fc-(Gly)₃-BM-lenalidomide bundles (i.e., “stabilized conjugates”), the results in FIGS. 12D and 12E indicated that the stabilized 2-chain (anti-CD38 scFv)-hIgG1.Fc-(Gly)₃-BM-lenalidomide bundle was at least 100 times more potent than lenalidomide alone or in combination with daratumumab. For U266-CD38-cells, the stabilized 2-chain (anti-CD38 scFv)-hIgG1.Fc-(Gly)₃-BM-lenalidomide bundles (i.e., “stabilized conjugates”), the 2-chain (anti-CD38 scFv)-hIgG1.Fc-(Gly)₃-BM (unconjugated, i.e., “anti-CD38 mAb”), and daratumumab did not possess any killing effect, whereas lenalidomide and its combination with daratumumab exhibited some killing effect at high concentrations likely due to the effect of lenalidomide (FIG. 12F). Although Daudi B lymphoma cells express CD38, all five treatments did not exhibit any killing effect as these cells are probably resistant to lenalidomide (FIG. 12G).

Furthermore, the stabilized 2-chain (anti-CD38 scFv)-hIgG1.Fc-(Gly)₃-BM-lenalidomide bundles exhibited desired toxicity to tumor cells via binding tightly to CD38 on the MM cell surface to form a complex of CD38 and the stabilized 2-chain (anti-CD38 scFv)-hIgG1.Fc-(Gly)₃-BM-lenalidomide bundle was efficiently internalized via endocytosis and cleaved by lysosomal cathepsin B to release cytotoxic lenalidomide, leading to cell death. Lenalidomide, daratumumab or their combination did not have this mechanism of action, in which lenalidomide unconjugated to daratumumab cannot easily enter cells, whereas daratumumab required Fc-dependent effector functions to kill CD38-expressing tumor cells. Hence, the stabilized 2-chain (anti-CD38 scFv)-hIgG1.Fc-(Gly)₃-BM-lenalidomide bundles possessed more potent cytotoxic effects in in vitro assays than lenalidomide, daratumumab, or their combination.

### Example 17 In Vitro Cytotoxic Activities of Native 2-Chain (Anti-CD38 scFv)-hIgG1.Fc-(Gly)₃-BM-Lenalidomide Bundles Through Native Lenalidomide Molecules Releasing

The assays of in vitro cytotoxicity activities of the native 2-chain (anti-CD38 scFv)-hIgG1.Fc-(Gly)₃-BM-lenalidomide bundles of Example 4 on the human multiple myeloma H929 and MM.1S cells were performed as described in above examples.

While using the stabilized 2-chain (anti-CD38 scFv)-hIgG1.Fc-(Gly)₃-BM-lenalidomide bundles (i.e., “stabilized conjugates”) as a positive control, the results of Table 2 indicated that the native 2-chain (anti-CD38 scFv)-hIgG1.Fc-(Gly)₃-BM-lenalidomide bundles (i.e., “native conjugates”) displayed potent cytotoxicities with the EC₅₀ values of 0.722 μM for treating with 2 hours and 0.207 UM for treating with 5 days on H929 cells.

TABLE 2
In vivo cytotoxicity of specified treatment in H929 cells

| Treatment | EC₅₀ (μM) |
|---|---|
| Stabilized conjugates for 2 hours | 3.698 |
| Stabilized conjugates for 5 days | 0.818 |
| Naive conjugates for 2 hours | 0.722 |
| Naive conjugates for 5 days | 0.207 |

Further, the results of Table 3 demonstrated that the native 2-chain (anti-CD38 scFv)-hIgG1.Fc-(Gly)₃-BM-lenalidomide bundles (i.e., “native conjugates”) displayed potent cytotoxicities with the EC₅₀ values of 0.661 μM for treating with 2 hours and 0.190 μM for treating 5 days on MM.1S cells.

TABLE 3
In vivo cytotoxicity of specified treatment in MM.1S cells

| Treatment | EC₅₀ (μM) |
|---|---|
| Stabilized conjugates for 2 hours | 1.685 |
| Stabilized conjugates for 5 days | 0.813 |
| Naive conjugates for 2 hours | 0.661 |
| Naive conjugates for 5 days | 0.190 |

### Example 18 Plasma Stability of Stabilized 2-Chain (Anti-CD38 scFv)-hIgG1.Fc-(Gly)₃-BM-Lenalidomide Bundles

To evaluate the plasma stability of the stabilized 2-chain (anti-CD38 scFv)-hIgG1.Fc-(Gly)₃-BM-lenalidomide bundles, the required amount of the 2-chain (anti-CD38 scFv)-hIgG1.Fc-(Gly)₃-BM (unconjugated antibody), the stabilized 2-chain (anti-CD38 scFv)-hIgG1.Fc-(Gly)₃-BM-lenalidomide bundles, or daratumumab was dissolved in 90% human plasma at a final concentration of 2 mg/mL, and then incubated at 37° C. for 28 days. A sample aliquot was collected and analyzed by ELISA on day 0, 3, 7, 10, 14, 21, and 28. The recombinant human CD38 proteins were coated as antigen to capture daratumumab, the 2-chain (anti-CD38 scFv)-hIgG1.Fc-(Gly)₃-BM (unconjugated antibody), and the stabilized 2-chain (anti-CD38 scFv)-hIgG1.Fc-(Gly)₃-BM-lenalidomide bundles; and a secondary antibody, HRP-conjugated anti-human IgG-Fc antibody, was used to detect the three CD38-binding antibodies. To determine whether the two lenalidomide bundles remained attached to the antibody of the stabilized 2-chain (anti-CD38 scFv)-hIgG1.Fc-(Gly)₃-BM-lenalidomide bundles, an anti-lenalidomide-bundle scFv-mouse IgG-Fc antibody that can be further detected by HRP-conjugated anti-mouse IgG-Fc antibody were used. A 96-well ELISA plate was coated with recombinant human CD38 protein (0.1 μg/100 μL/well) for 16 hours at 4° C. After washing with PBST buffer (PBS with 0.1% TWEEN® 20), each well was blocked with the blocking buffer (PBS with 1% bovine serum albumin) for 30 minutes. The standards were prepared by dissolving the stabilized 2-chain (anti-CD38 scFv)-hIgG1.Fc-(Gly)₃-BM-lenalidomide bundles with human plasma and diluting it in blocking buffer. All samples were prepared by diluting in a blocking buffer to match the linearity of the calculation curve. The standards and diluted samples were added to each well and incubated at room temperature for 1.5 hours. After washing thrice with PBST, the secondary antibodies with proper dilution in the blocking buffer were added to the corresponding well. To detect the stabilized 2-chain (anti-CD38 scFv)-hIgG1.Fc-(Gly)₃-BM-lenalidomide bundles, HRP-conjugated anti-mouse IgG-Fc antibody was further added after washing off the anti-lenalidomide-bundle scFv-mouse IgG-Fc antibody. Finally, the concentration of each antibody was quantified using a tetramethylbenzidine reagent and microplate reader. The half-life (T_{1/2}) was calculated by software. Data were shown as mean±SD.

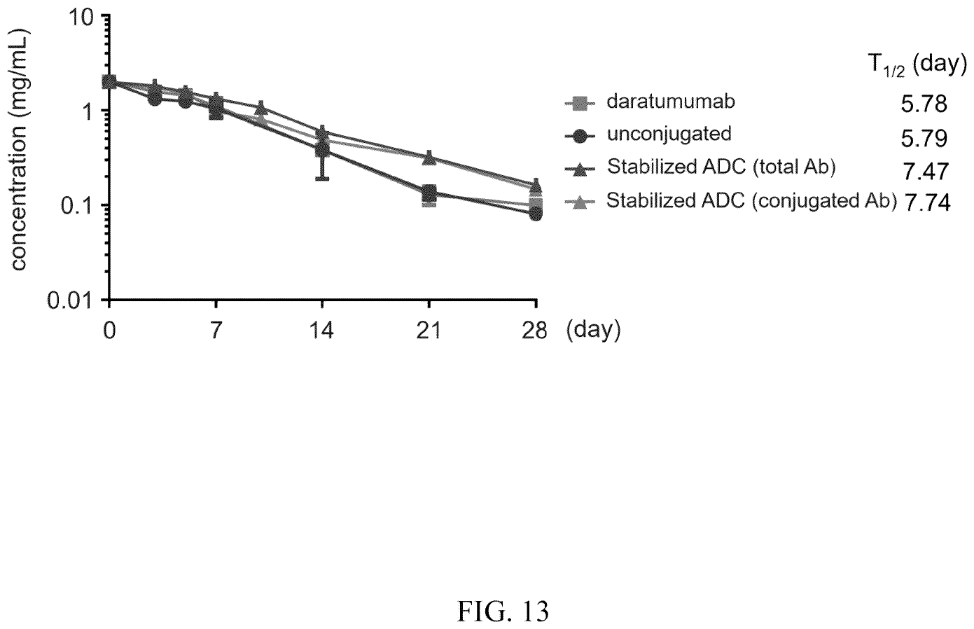

The results depicted in FIG. 13 indicated that daratumumab, the 2-chain (anti-CD38 scFv)-hIgG1.Fc-(Gly)₃-BM fusion protein (labeled as “unconjugated”), and the stabilized 2-chain (anti-CD38 scFv)-hIgG1.Fc-(Gly)₃-BM-lenalidomide bundles shared a comparable half-life (T_{1/2}) of about 6-7 days in human plasma for 28 days. Since the secondary antibody, i.e., HRP-conjugated anti-human IgG-Fc antibody, cannot distinguish between the stabilized 2-chain (anti-CD38 scFv)-hIgG1.Fc-(Gly)₃-BM-lenalidomide bundles and the 2-chain (anti-CD38 scFv)-hIgG1.Fc-(Gly)₃-BM fusion protein conjugated to ≤1 lenalidomide bundle, an anti-lenalidomide-bundle scFv-mouse IgG-Fc antibody, which was detected by a secondary antibody, i.e., HRP-conjugated anti-mouse IgG-Fc antibody, was used to determine whether the two lenalidomide bundles remained attached to the anti-CD38 antibody. The concentration of the lenalidomide-bundle conjugated antibody at various time points indicated that the T_{1/2} (7.7 days) thereof (labeled as “stabilized ADC (conjugated Ab), i.e., the 2-chain (anti-CD38 scFv)-hIgG1.Fc-(Gly)₃-BM-lenalidomide bundles detected by the anti-lenalidomide bundle antibody and HRP-conjugated anti-mouse IgG-Fc antibody) was nearly identical to the Tin (7.5 days) of total stabilized 2-chain (anti-CD38 scFv)-hIgG1.Fc-(Gly)₃-BM-lenalidomide bundles (labeled as “stabilized ADC (total Ab)” that contained both the 2-chain (anti-CD38 scFv)-hIgG1.Fc-(Gly)₃-BM-lenalidomide bundles and the 2-chain (anti-CD38 scFv)-hIgG1.Fc-(Gly)₃-BM fusion protein conjugated to ≤1 lenalidomide bundle) detected by the HRP-conjugated anti-human IgG-Fc antibody (FIG. 13). This implies that the 2 lenalidomide bundles were stably conjugated on the anti-CD38 antibody in human plasma. Furthermore, despite the reduced scFv of the anti-CD38 antibody, its Tin was not reduced compared to daratumumab's T_{1/2}.

### Example 19 Establishment of In Vivo Tumor Models of Stabilized 2-Chain (Anti-CD38 scFv)-hIgG1.Fc-(Gly)₃-BM-Lenalidomide Bundles

To generate subcutaneous tumors, 2×10⁷ H929 cells in PBS were mixed with 50% (v/v) extracellular matrix gel and injected subcutaneously into the flanks of NOD-SCID mice. Tumor size and body weight were recorded every 2-3 days. Volumes of subcutaneously xenografted tumors in vivo were determined using an external caliper, where tumor volume was calculated by the modified ellipsoid formula, ½×(length×width×width). When the average size of tumor was in the range of 115±15 mm³ (7 days after implantation) or 150±20 mm³ (14 days after implantation), the stabilized 2-chain (anti-CD38 scFv)-hIgG1.Fc-(Gly)₃-BM-lenalidomide bundles, the 2-chain (anti-CD38 scFv)-hIgG1.Fc-(Gly)₃-BM (unconjugated antibody) and daratumumab were respectively prepared in PBS and intraperitoneally (i.p.) administered to the mice. In all of experiments, a single dose (20 nmol/kg; about 2.3 mg/kg) of the stabilized 2-chain (anti-CD38 scFv)-hIgG1.Fc-(Gly)₃-BM-lenalidomide bundles or daratumumab was administered. At the end of experiments, tumors were collected and weighed. Data were analyzed by software, and expressed as mean±SD or mean±SEM as indicated in the figures. Statistical analysis was performed with Student t test and statistical differences between groups were analyzed by one-way ANOVA with Bonferroni's multiple comparison post-tests.

### Example 20 Anti-Tumor Efficacy of Stabilized 2-Chain (Anti-CD38 scFv)-hIgG1.Fc-(Gly)₃-BM-Lenalidomide Bundles and Daratumumab in Mouse Xenograft Tumor Model

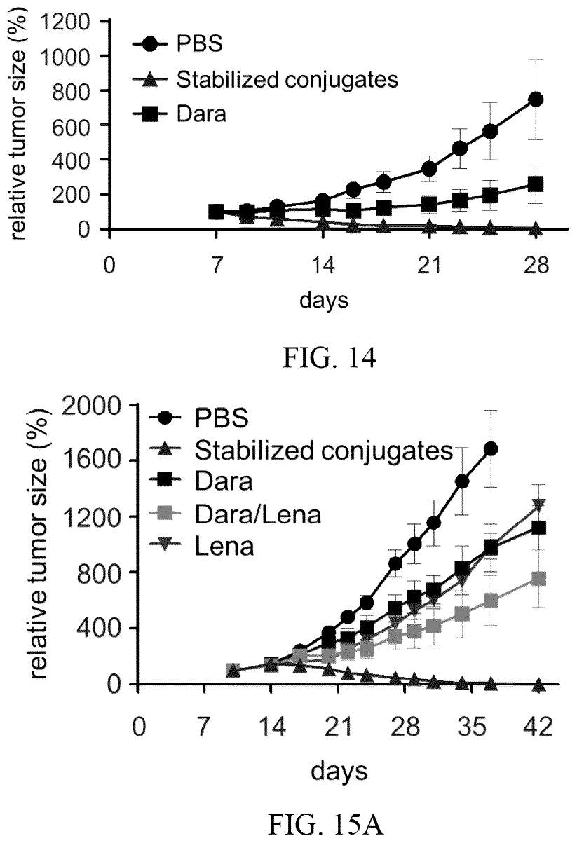

In the pilot study using H929 cells, a single dose (20 nmol/kg) of the stabilized 2-chain (anti-CD38 scFv)-hIgG1.Fc-(Gly)₃-BM-lenalidomide bundles or daratumumab was administered i.p. 7 days post-transplantation, when the tumors grew to an average size of 115±15 mm³. The changes of tumor sizes measured every 2-3 days for 21 days demonstrated that the stabilized 2-chain (anti-CD38 scFv)-hIgG1.Fc-(Gly)₃-BM-lenalidomide bundle was capable of effectively shrinking tumor sizes, while daratumumab (labeled as “Dara”) could only slow down tumor growth, as compared to PBS (FIG. 14). The tumors from all mice were excised and weighed on day 21 post drug administration. The results indicated that the stabilized 2-chain (anti-CD38 scFv)-hIgG1.Fc-(Gly)₃-BM-lenalidomide bundles (labeled as “stabilized conjugates”) exhibited much better growth inhibitory activity against H929 tumors compared to daratumumab.

### Example 21 Anti-Tumor Efficacy of the Stabilized 2-Chain (Anti-CD38 scFv)-hIgG1.Fc-(Gly)₃-BM-Lenalidomide Bundles, Daratumumab, Lenalidomide, and Daratumumab/Lenalidomide Combination in Mouse Xenograft Tumor Model

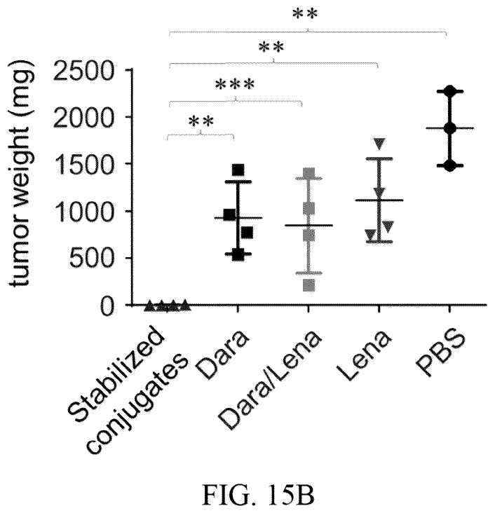

The stabilized 2-chain (anti-CD38 scFv)-hIgG1.Fc-(Gly)₃-BM-lenalidomide bundle was subsequently studied for its ability to inhibit tumor growth, when the tumors were allowed additional days to grow larger. On day 14 after the transplantation of H929 cells, when the tumors reached an average volume of approximately 150±20 mm³, the mice were respectively administered with (1) a single-dose of daratumumab (20 nmol/kg), (2) daily i.p. injections of lenalidomide (46 mmol/kg/day), (3) a daratumumab/lenalidomide combo (a single-dose of 20 nmol/kg of daratumumab+46 mmol/kg/day of lenalidomide), and (4) a single-dose of the stabilized 2-chain (anti-CD38 scFv)-hIgG1.Fc-(Gly)₃-BM-lenalidomide bundles (labeled as “stabilized conjugates”) (20 nmol/kg; about 2.3 mg/kg). The results of FIG. 15A demonstrated that compared to PBS treatment, the treatment of daratumumab (Dara) or lenalidomide (Lena) inhibited tumor growth, and the daratumumab/lenalidomide combo (Dara/Lena) treatment exhibited a greater inhibitory effect on tumor growth. The tumors from all mice were excised and weighed on day 28 post drug administration (FIG. 15B). Surprisingly, the treatment of the stabilized 2-chain (anti-CD38 scFv)-hIgG1.Fc-(Gly)₃-BM-lenalidomide bundles (stabilized conjugates) completely suppressed tumor growth in the mice, leaving a minuscule nodule in one of the mice (FIGS. 15A and 15B). The results indicated that the stabilized 2-chain (anti-CD38 scFv)-hIgG1.Fc-(Gly)₃-BM-lenalidomide bundles exhibited much better anti-tumor effect on H929 tumors as compared to that of daratumumab, lenalidomide, or daratumumab/lenalidomide combo treatment.

Notably, the total amount of lenalidomide employed in the 46 mmol/kg/day treatment over the course of 28 days was 10,640 times the amount of lenalidomide provided by a single dose (20 nmol/kg) of the stabilized 2-chain (anti-CD38 scFv)-hIgG1.Fc-(Gly)₃-BM-lenalidomide bundles.

### Example 22 Anti-tumor efficacy of the stabilized 2-chain (anti-CD38 scFv)-hIgG1.Fc-(Gly)₃-BM-Lenalidomide Bundles and Daratumumab/Lenalidomide Combination Using Patient-Derived Multiple Myeloma Cells

To evaluate the effectiveness of the stabilized 2-chain (anti-CD38 scFv)-hIgG1.Fc-(Gly)₃-BM-lenalidomide bundles in patient-derived multiple myeloma (MM) cells, MM cells isolated from the bone marrow of five newly diagnosed MM patients (No. 1 to No. 5) were treated with various concentrations (0.00064, 0.0032, 0.016, 0.08, 0.4, 2, 10 UM) of the stabilized 2-chain (anti-CD38 scFv)-hIgG1.Fc-(Gly)₃-BM-lenalidomide bundles or a combination of daratumumab and lenalidomide (i.e., daratumumab/lenalidomide combination; serving as the control group) for 5 days at 37° C. Cell viability was determined using alamarBlue™ cell viability reagent.

The results of the cell viability of MM cells treated with the stabilized 2-chain (anti-CD38 scFv)-hIgG1.Fc-(Gly)₃-BM-lenalidomide bundles revealed a significant decrease in viability. The half-maximal inhibitory concentration (IC₅₀) values for the stabilized 2-chain (anti-CD38 scFv)-hIgG1.Fc-(Gly)₃-BM-lenalidomide bundles ranged from 0.07 to 0.27 μM, and the IC₅₀ values for each combination treatment group exceeded 100 μM (Table 4).

TABLE 4
IC₅₀ value of specified treatments

IC₅₀ (μM)

| | Stabilized 2-chain (anti-CD38 scFv)- | Daratumumab/ |
|---|---|---|
| Patient | hIgG1.Fc-(Gly)₃-BM-lenalidomide | lenalidomide |
| No. | bundles | combination |
| 1 | 0.100 | >100 |
| 2 | 0.273 | >100 |
| 3 | 0.094 | >100 |
| 4 | 0.195 | >100 |
| 5 | 0.077 | >100 |

Furthermore, the analysis of IC₅₀ values demonstrated a more than 100-fold decrease in IC₅₀ values with the stabilized 2-chain (anti-CD38 scFv)-hIgG1.Fc-(Gly)₃-BM-lenalidomide bundles as compared to free lenalidomide in the daratumumab/lenalidomide combination treatment (Table 4). This result indicates that lenalidomide carried by the stabilized conjugates is significantly more effective in killing MM cells, emphasizing its potency once internalized by MM cells.

The results suggest that the stabilized 2-chain (anti-CD38 scFv)-hIgG1.Fc-(Gly)₃-BM-lenalidomide bundles exhibit potent anti-tumor efficacy in patient-derived MM cells. The significant decrease in IC₅₀ values compared to free lenalidomide highlights the enhanced effectiveness of the lenalidomide bundle conjugation.

In summary, the stabilized 2-chain (anti-CD38 scFv)-hIgG1.Fc-(Gly)₃-BM-lenalidomide bundles exhibited more potent cytotoxic effects in in vivo assays than lenalidomide, daratumumab, or their combination.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method of treating a tumor in a subject, comprising administering to the subject a molecular construct comprising an anti-CD38 antibody, and a plurality of lenalidomide molecules or hydrolyzed lenalidomide molecules linked to the anti-CD38 antibody, wherein the administration of the molecular construct gives rise to an effective amount of the lenalidomide molecules or the hydrolyzed lenalidomide molecules that is at least 1,000 times less than an effective amount of the lenalidomide molecule used alone or in combination with the anti-CD38 antibody for the treatment of the tumor.

2. The method of claim 1, wherein the effective amount of the lenalidomide molecules or the hydrolyzed lenalidomide molecules is about 10,000 times less than the effective amount of the lenalidomide molecule used alone or in combination with the anti-CD38 antibody for the treatment of the tumor.

3. The method of claim 1, wherein the molecular construct is administered to the subject in an amount of about 0.1-10 mg/Kg.

4. The method of claim 3, wherein the molecular construct is administered to the subject once every four weeks.

5. The method of claim 1, wherein the anti-CD38 antibody comprises,
a pair of CH2-CH3 segments of an immunoglobulin G (IgG), wherein the pair of CH2-CH3 segments comprises a plurality of linking residues independently selected from the group consisting of lysine (K) and cysteine (C) residues; and
a pair of anti-CD38 single-chain variable fragments (scFvs) respectively linked to the N-termini of the pair of CH2-CH3 segments;
wherein the plurality of lenalidomide molecules are respectively linked to the plurality of linking residues.

6. The method of claim 1, wherein the anti-CD38 antibody comprises,
a pair of CH2-CH3 segments of an IgG;
a pair of anti-CD38 scFvs respectively linked to the N-termini of the pair of CH2-CH3 segments; and
a pair of linking peptides respectively linked to the C-termini of the pair of CH2-CH3 segments, wherein the pair of linking peptides comprises a plurality of C residues;
wherein the plurality of lenalidomide molecules are respectively linked to the plurality of C residues of the pair of linking peptides.

7. The method of claim 6, wherein each of the pair of linking peptides comprises the amino acid sequence of “CGGHA” (SEQ ID NO: 1), “CPGHA” (SEQ ID NO: 2), “CGAHA” (SEQ ID NO: 3), “CPAHA” (SEQ ID NO: 4), “GCGGHA” (SEQ ID NO: 5), “ACPGHA” (SEQ ID NO: 6), or “GCPGHA” (SEQ ID NO: 7).

8. The method of claim 7, wherein each of the pair of linking peptides comprises the amino acid sequence of “ACPGHA” (SEQ ID NO: 6).

9. The method of claim 6, wherein the molecular construct further comprises a linker unit, which comprises,
a center core that is in a linear form and comprises, 2 to 10 K residues;
at least one filler independently disposed between two K residues; and
a terminal spacer having two termini, in which one of the termini is linked to the N-terminus of the first K residue or the C-terminus of the last K residue, and the other of the termini is linked to the C residue of the linking peptide of the anti-CD38 antibody;
wherein each of the filler and the terminal spacer independently comprises, (1) 1 to 12 non-K amino acid residues, or (2) a PEGylated amino acid having 1 to 12 repeats of ethylene glycol (EG) unit; and
2 to 10 linking arms, wherein one terminus of each linking arm is linked to one of the K residues of the center core, and the other terminus of each linking arm is linked to each lenalidomide molecule or hydrolyzed lenalidomide molecule.

10. The method of claim 9, wherein the terminal spacer comprises at least three negative charged amino acid residues.

11. The method of claim 10, wherein the terminal spacer comprises the amino acid sequence of “EDEDEAGG” (SEQ ID NO: 8), “EGEGEAGG” (SEQ ID NO: 9) or “EGEGE” (SEQ ID NO: 10.

12. The method of claim 11, wherein the center core comprises the amino acid sequence of “EDEDEGAGGKGAGKGAGKG” (SEQ ID NO: 11).

13. The method of claim 9, wherein each of the linking arms comprises 2-12 non-K amino acid residues, a polyethylene glycol (PEG) chain having 2-24 repeats of EG units, or a combination thereof.

14. The method of claim 9, wherein each of the linking arms is linked to the &-amino group of the K residue.

15. The method of claim 1, wherein the tumor is a solid tumor or a diffused tumor.

16. The method of claim 15, wherein the solid tumor is melanomas, esophageal carcinomas, gastric carcinomas, brain tumor, small cell lung cancer, non-small cell lung cancer, bladder cancer, breast cancer, pancreatic cancer, colon cancer, rectal cancer, colorectal cancer, renal cancer, hepatocellular carcinoma, ovary cancer, prostate cancer, thyroid cancer, testis cancer, or head and neck squamous cell carcinoma.

17. The method of claim 15, wherein the diffused tumor is acute lymphocytic leukemia (ALL), chronic lymphocytic leukemia (CLL), acute myelogenous leukemia (AML), chronic myelogenous leukemia (CML), Hodgkin lymphoma, non-Hodgkin lymphoma, or multiple myeloma.
